(19) United States
(12) Patent Application Publication
(10) Pub. No.: US 2008/0113337 A1
(43) Pub. Date: May 15, 2008

(54) Method of Examining/Judging Presence of Virus Infection such as HIV or Presence of Prion Infection by Near-Infrared Spectroscopy and Device Used in Same

(75) Inventors: Akikazu Sakudo, Osaka (JP); Roumiana Tsenkova, Hyogo (JP); Kazuyoshi Ikuta, Osaka (JP); Takashi Onodera, Ibaraki (JP)

Correspondence Address: KIRK HAHN, 14431 HOLT AVE, SANTA ANA, CA, 92705, US

(73) Assignees: THE NEW INDUSTRY RESEARCH ORGANIZATION, Kobe (JP); OSAKA UNIVERSITY, Osaka (JP)

(21) Appl. No.: 11/718,980

(22) PCT Filed: Nov. 10, 2005
(86) PCT No.: PCT/JP05/20595

§ 371 Date: Sep. 28, 2007

(30) Foreign Application Priority Data
Nov. 12, 2004 (JP) 2004-329249

(51) Int. Cl. C12Q 1/70; A61B 5/1455

(52) U.S. Cl. 435/5; 600/315

(57) ABSTRACT

The present invention provides a method and device of quantitatively or qualitatively examining and judging the presence of virus infection, such as HIV, or the presence of prion infection by: irradiating a sample derived from an examinee or other animal with light having a wavelength in a range of 400 nm to 2500 nm or a wavelength in part of the range; detecting reflected light, transmitted light, or transmitted and reflected light to obtain an absorption spectral data; and analyzing the absorbance at all measurement wavelengths or at specific wavelengths in the absorption spectral data by using an analytical model prepared beforehand. The analytical model can be prepared by carrying out spectral measurement, with a perturbation being provided, and carrying out a multivariate analysis that brings out perturbation effects.

### TECHNICAL FIELD

The present invention relates to methods of examining and judging the presence of virus infection, such as HIV, or the presence of prion infection by near-infrared spectroscopy and devices that are used in these methods.

### BACKGROUND ART

Currently, virus infection tests, such as for HIV (human immunodeficiency virus) and HCV (hepatitis C virus), are mainly performed using as an indicator (1) the detection of virus DNA by a PCR method, or (2) detection of an antiviral antibody or a viral antigen, for instance, by an ELISA method (enzyme-linked immunosorbent assay). As an example, in an HIV infection test a method is employed in which the presence of an HIV p24 antigen is detected by the ELISA method or Western blotting (see Nonpatent document 1, described later).

However, this method requires complicated processing and takes time. Therefore a demand exists for a simple and quick method of examining and diagnosing virus infection.

On the other hand, mainly the detection of abnormal prion protein (PrPSC) by an immunohistochemical method or the Western blotting or ELISA method with an anti-prion protein antibody is used for the test of prion infection and the diagnosis of prion diseases. A bioassay carried out through animal inoculation is also employed in research (see Nonpatent document 2, described later). However, these methods are all postmortem tests. They also require complicated processing and take time. Therefore demands exist for a simple and quick antemortem examination and diagnostic methods in prion detection.

Currently, a componential analysis using near-infrared rays is being carried out in various fields. For example, a sample is irradiated with visible light and/or near-infrared rays, a wavelength range in which the visible light and/or near-infrared rays are absorbed by a specific component is detected, and the specific component is then analyzed quantitatively.

This is carried out as follows: For example, a sample is injected into a quartz cell, and this is irradiated with visible light and/or near-infrared rays in a wavelength range of 400 to 2500 nm, using a near-infrared spectroscope (such as an NTRSystem 6500 manufactured by Nireco Corp.); transmitted light, reflected light, and transmitted and reflected light are analyzed.

Generally, near-infrared rays have a very small absorbance coefficient to a substance, hardly undergo scattering, and are also a low-energy electromagnetic wave. Therefore they allow chemical/physical data to be obtained without damaging the sample.

Thus sample data can be obtained immediately by detecting, for example, transmitted light from a sample, determining the absorbance data of the sample, and subjecting this data to a multivariate analysis. For instance, the biomolecular structure or the process of metergasia can be obtained directly and in real time.

Examples of the conventional techniques relating to such near-infrared spectroscopy include those described in patent documents 1 and 2 below. Patent document 1 discloses a method of obtaining data from a sample using visible to near-infrared rays, specifically, a method of discriminating the group to which an unknown sample belongs, a method of identifying an unknown sample, and a method of monitoring the time-dependent change in the sample in real time. This document does not disclose the virus detection and prion detection carried out by near-infrared spectroscopy.

Patent document 2 discloses a method of diagnosing bovine mastitis by measuring a somatic cell in milk or the udder through a multivariate analysis of absorbance data obtained, using an absorption band of water molecules in the visible light and/or near-infrared region.

Furthermore, patent document 3 discloses a method of diagnosing the change induced by transmissible spongiform encephalopathy (TSE) in tissues of an animal or a human by measuring their infrared spectra. This method also is carried out using a postmortem histopathologic piece as a test object and is thus a postmortem test.

[Nonpatent document 1] Valdiserri R O, Holtgrave D R, West G R. Promoting early HIV diagnosis and entry into care. AIDS. 1999 13(17):2317-30.

[Nonpatent document 2] Aguzzi A, Heikenwalder M, Miele G. Progress and problems in the biology, diagnostics, and therapeutics of prion diseases. J Clin Invest. 2004 114(2):153-60.

[Patent document 1] Japanese Laid-Open Patent Publication No. 2002-5827 (pp. 1-9, FIG. 1)

[Patent document 2] International Publication No. WO 01/75420 (pp. 1-5, FIG. 1)

[Patent document 3] Published Japanese translation of PCT international application No. 2003-500648.

### DISCLOSURE OF INVENTION

As described above, there are demands for simple, quick, and highly accurate examination and diagnostic methods in the diagnosis of viral infectious diseases, such as an HIV or HCV test. Particularly, for example, when it is necessary to examine a large quantity of samples simultaneously, there are high demands for the development of a simple and quick examination method. In a virus test for donated blood, for example, it is desirable to develop a method for simply, quickly, and inexpensively testing the presence of such virus as HIV, HBV, or HCV in a large quantity of samples. Currently, in advanced nations and especially in Japan, virus tests of donated blood are carried out mainly by antibody tests. On the other hand, virus tests in developing countries are carried out only in some areas, and only part of the donated blood is examined.

Therefore the present invention is intended to provide a novel method and device for quantitatively or qualitatively examining and judging the presence of virus infection, such as HIV, in a sample simply, quickly, and with high accuracy, using a near-infrared spectroscopy.

Similarly, in the detection of prion and the diagnosis of a prion disease, there are demands for simple, quick, and not postmortem tests, but antemortem tests and diagnostic methods. Especially, for example, when it is necessary to examine a large quantity of samples simultaneously for prion diseases in domestic animals, such as bovine spongiform encephalopathy (mad cow disease) and scrapie, the development of such a simple and quick examination method is highly demanded. Furthermore, two cases of variant CJD infection caused by blood transfusion in Great Britain have been recently reported, and this has become a social problem. Since transmission through blood was also found in an experiment using sheep, there is currently a concern about the possibility of CJD infection through blood transfusion. Therefore a method to simply and quickly examine prion from blood is demanded.

The present invention is further intended to provide a novel method and device for quantitatively or qualitatively examining and judging the presence of prion infection in a sample simply, quickly, and with high accuracy, using near-infrared spectroscopy.

With the aforementioned problems in mind, the present inventors devoted themselves to studies. As a result, they have discovered the following. It is possible, for example, to examine and diagnose prion infection and virus infection, such as HIV by near-infrared spectroscopy, particularly a method of measuring visible light—near-infrared ray (VIS-NIR) spectra. The preparation of an analytical model through a creative method of analyzing resultant spectral data would allow excellent examinations and diagnoses to be carried out with its use. Thus the present invention was completed.

That is, the present invention embraces the following as medically and industrially useful inventions:

(A) A method of quantitatively or qualitatively examining and judging the presence of virus infection, such as HIV, or the presence of prion infection, with the method including:
irradiating a sample derived from an examinee or other animal with light having a wavelength in a range of 400 nm to 2500 nm or a wavelength in part of the range,
detecting reflected light, transmitted light, or transmitted and reflected light to obtain an absorption spectral data, and
analyzing the absorbance at all measurement wavelengths or at specific wavelengths in the absorption spectral data by using an analytical model prepared beforehand;

(B) The examining and judging method according to item (A) described above, wherein a spectrum is measured, with a perturbation being provided for the sample by adding a predetermined condition, and an analysis that brings out an effect of the perturbation is performed;

(C) The examining and judging method according to item (B) described above, wherein the predetermined condition is any one of the following: change in concentration (including concentration dilution), repeated light irradiation, extension of irradiation time, addition of electromagnetic power, change in light path length, temperature, pH, pressure, mechanical vibration, and one that brings about physical or chemical change by changing the condition, or a combination thereof;

(D) The examining and judging method according to item (C) described above, wherein light irradiation is repeated three times and thereby three absorption spectral data are obtained, and then a multivariate analysis is carried out by using at least two of the three absorption spectral data;

(E) The examining and judging method according to item (C) described above, wherein with respect to one sample, those having a plurality of concentrations are prepared by dilution, they each are subjected to spectrum measurement, and a multivariate analysis is carried out by using the spectrum data thus obtained;

(F) The examining and judging method according to any one of items (A) to (E) described above, wherein the method is used for an HIV or prion test, and a target substance in a sample, such as HIV p24, is quantified by using a quantitative model prepared by a regression analysis, such as a PLS method;

(G) The examining and judging method according to any one of items (A) to (E) described above, wherein the method is used for an HIV or prion test, and the presence of infection or a further infection period is estimated using a qualitative model prepared by a class discriminant analysis, such as a SIMCA method;

(H) The examining and judging method according to any one of items (B) to (E) described above, wherein the method is used for an HIV or prion test, and the presence of infection or a further infection period is estimated using a qualitative model prepared by (1) carrying out a regression analysis such as a PLS method in which each value of perturbation such as a value of change in concentration is used as a dependent variable, and (2) carrying out a class discriminant analysis such as a SIMCA method with respect to a regression vector obtained by the regression analysis;

(I) The examining and judging method according to any one of items (A) to (H) described above, wherein the wavelength region of the light with which the sample is irradiated is set in a range that is required for an analysis to be carried out using an analytical model;

(J) The examining and judging method according to item (I) described above, wherein the wavelength region of the light with which the sample is irradiated is set in a range of 600 nm to 1000 nm;

(K) The examining and judging method according to any one of items (A) to (J), wherein the sample is blood (including blood plasma and serum), urine, another biological fluid, a tissue, a tissue extract, or a biological part such as an ear, an abdomen, or a fingertip of a hand or foot;

(L) A test and diagnostic device, including:
a floodlight means for irradiating a sample derived from an examinee or other animal with light having a wavelength in a range of 400 nm to 2500 nm or a wavelength in part of the range,
a spectroscopic means for carrying out spectroscopy before or after irradiation and a detection means for detecting reflected light, transmitted light, or transmitted and reflected light with respect to the light with which the sample was irradiated, and
a data analysis means for analyzing quantitatively or qualitatively the presence of virus infection, such as HIV, or the presence of prion infection by analyzing the absorbance at all measurement wavelengths or at specific wavelengths in the absorption spectral data obtained by the detecting, using an analytical model prepared beforehand;

(M) The test and diagnostic device according to item (L) described above, further including a display means for displaying a result of the analysis;

(N) The test and diagnostic device according to item (L) described above, wherein a spectrum is measured, with a perturbation being provided for the sample by adding a predetermined condition, and an analysis that brings out an effect of the perturbation is performed;

(O) The test and diagnostic device according to item (N) described above, wherein the predetermined condition is any one of the following: change in concentration (including concentration dilution), repeated light irradiation, extension of irradiation time, addition of electromagnetic power, change in light path length, temperature, pH, pressure, mechanical vibration, and one that brings about physical or chemical change by changing the condition, or a combination thereof;

(P) The test and diagnostic device according to item (0) described above, wherein light irradiation is repeated three times and thereby three absorption spectral data are obtained, and a multivariate analysis is then carried out by using at least two of the three absorption spectral data;

(Q) The test and diagnostic device according to any one of items (L) to (P) described above, wherein the test and diagnostic device is used for an HIV or prion test, and a target substance in a sample, such as HIV p24, is quantified by using a quantitative model prepared by a regression analysis such as a PLS method;

(R) The test and diagnostic device according to any one of items (L) to (P) described above, wherein the test and diagnostic device is used for an HIV or prion test, and the presence of infection or a further infection period is estimated using a qualitative model prepared by a class discriminant analysis such as a SIMCA method;

(S) The test and diagnostic device according to any one of items (N) to (P) described above, wherein the test and diagnostic device is used for an HIV or prion test, and the presence of infection or a further infection period is estimated using a qualitative model prepared by (1) carrying out a regression analysis such as a PLS method in which each value of perturbation such as a value of change in concentration is used as a dependent variable, and (2) carrying out a class discriminant analysis such as a SIMCA method with respect to a regression vector obtained by the regression analysis;

(T) The test and diagnostic device according to any one of items (L) to (S) described above, wherein the wavelength region of the light with which the sample is irradiated is set in a range required for an analysis to be carried out using an analytical model;

(U) The test and diagnostic device according to item (T) described above, wherein the wavelength region of the light with which the sample is irradiated is set in a range of 600 nm to 1000 nm; and

(V) The test and diagnostic device according to any one of items (L) to (U) described above, wherein the sample is blood (including blood plasma and serum), urine, another biological fluid, a tissue, a tissue extract, or a biological part such as an ear, an abdomen, or a fingertip of a hand or foot.

The present invention makes it possible to simply, quickly, and highly accurately examine and judge the presence of prion infection and virus infection, such as HIV, and therefore it can be used widely for various virus tests and the prion test. Since it can be carried out simply and quickly, it is useful, for example, when a large quantity of samples is required to be examined simultaneously. Furthermore, it allows a target substance in a sample to be quantified with high accuracy.

The present invention also allows various virus tests and the prion test to be carried out by using a sample derived from blood, such as plasma or serum. Thus the present invention can be carried out simply and quickly and is also applicable to an antemortem diagnosis of a prion disease.

Examples of the sample to be used can include urine, another biological fluid, a tissue (mass of tissue), and a tissue extract (tissue homogenate), besides blood. Furthermore, it also is possible to test without damaging a biological body, by using as a sample a biological part such as an ear, an abdomen, or a fingertip of a hand or foot.

### BRIEF DESCRIPTION OF DRAWINGS

FIG. 1 is a block diagram showing a schematic configuration of a device according to an embodiment.

FIG. 2 is a diagram illustrating two spectroscopic methods, pre- and postspectroscopy, that can be employed in the aforementioned device.

FIG. 3 is a diagram illustrating three detection methods, reflected light detection, transmitted and reflected light detection, and transmitted light detection, that can be employed in the aforementioned device.

FIG. 4 is a diagram for explaining a suitable spectral measurement method and a data analytical method in the present invention.

FIG. 5 is a diagram illustrating the progress after HIV infection.

FIG. 6 shows graphs illustrating a Coomans plot obtained by a SIMCA analysis of 13 samples, each of which had been diluted 10 times in an HIV test.

FIG. 7 is a graph showing, with respect to the interclass distance, the results of a SIMCA analysis of 13 samples that had been diluted 10¹ to 10¹⁰ times to have different concentrations from one another in an HIV test.

FIG. 8 is a graph showing the results of factor selections in a PLS regression analysis that was carried out for preparing an analytical model to estimate the amount of HIV p24 in a sample.

FIG. 9 is a graph showing the results of the PLS regression analysis, along with the actual values (horizontal axis) and estimated values (vertical axis) of p24 amounts by comparison.

FIG. 10 is a graph showing the results obtained by carrying out the aforementioned PLS regression analysis, along with all partial regression coefficients (regression vectors) of a multiple regression type prepared as a quantification model.

FIG. 11 is a graph showing the results of factor selections with respect to Sample 1.

FIG. 12 shows the results of the PLS regression analysis carried out with respect to Sample 1, with the rate of dilution of the sample being indicated in the X axis, and with the values estimated with the quantitative model obtained by the analysis in regard to the respective values of the rate of dilution being plotted in the Y axis.

FIG. 13 shows the results of the PLS regression analysis carried out with respect to Sample 1 and is a graph showing all partial regression coefficients (regression vectors) of a multiple regression type prepared as a quantitative model.

FIG. 14 is a graph showing the respective regression vectors of Samples 1 to 5 that belong to Class 1, by comparison.

FIG. 15 is a graph showing the respective regression vectors of Samples 7, 9, 10, and 13 that belong to Class 2, by comparison.

FIG. 16 is a graph showing the respective regression vectors of Samples 6, 8, 11, and 12 that belong to Class 3, by comparison.

FIG. 17 is a graph showing the discriminating power (vertical axis) at each wavelength (horizontal axis) that was obtained as a result of the SIMCA analysis carried out with the regression vectors being considered as spectra.

FIG. 18 is a graph showing the discriminating power (vertical axis) at each wavelength (horizontal axis) that was obtained as a result of the same SIMCA analysis as in FIG. 17, except for Samples 12 and 13.

FIG. 19 is a graph showing, with respect to the interclass distance, the results of the SIMCA analysis carried out by using one of three absorption data obtained through three consecutive times of irradiation, or at least two of them.

FIG. 20 is a graph showing a Coomans plot obtained by the SIMCA analysis through a measurement of near-infrared absorption spectra of blood collected from a wild-type mouse, a prion protein gene knockout mouse, and a prion-infected wild-type mouse.

FIG. 21 is a graph showing a Coomans plot obtained by the SIMCA analysis through a measurement of near-infrared absorption spectra of brain tissues collected from a wild-type mouse, a prion protein gene knockout mouse, and a prion-infected wild-type mouse.

FIG. 22 is a graph showing a Coomans plot obtained by the SIMCA analysis through a measurement of near-infrared absorption spectra of brain homogenate prepared from a wild-type mouse, a prion protein gene knockout mouse, and a prion-infected wild-type mouse.

FIG. 23 is a graph showing a Coomans plot (Factor 40) of models for discriminating between prion infection and noninfection on and after 170 days from the inoculation, with the model being prepared by the SIMCA analysis through the measurements of near-infrared absorption spectra over time from the respective ears of a Chandler-strain-inoculated mouse, an Obihiro-strain-inoculated mouse, a normal-brain-homogenate-inoculated mouse, and a PBS-inoculated mouse.

FIG. 24 is a graph that shows the results estimated from the above-mentioned discrimination models obtained through measurements from the ears, and the percentage (%) at which the prion-infected mice subjected to measurements over time are diagnosed to be prion-infected using the models.

FIG. 25 is a graph showing a discriminating power (Factor 40) at each wavelength with regard to prion infection and noninfection in the discrimination models obtained through measurements from the ears.

FIG. 26 is a graph showing a Coomans plot (Factor 60) of models for discriminating between prion infection and noninfection on and after 170 days from the inoculation, which are prepared by the SIMCA analysis through the measurement of near-infrared absorption spectra over time from the respective abdomens of a Chandler-strain-inoculated mouse, an Obihiro-strain-inoculated mouse, a normal-brain-homogenate-inoculated mouse, and a PBS-inoculated mouse.

FIG. 27 is a graph that shows the result estimated from the above-mentioned discrimination models obtained through the measurement from the abdomens, and the percentage (%) at which the prion-infected mice subjected to the measurements over time are diagnosed to be prion-infected using the models.

FIG. 28 is a graph showing a discriminating power (Factor 60) at each wavelength with regard to prion infection and noninfection in the discrimination models obtained through measurements from the abdomens.

### DESCRIPTION OF THE PREFERRED EMBODIMENTS

Hereinafter, a device (hereinafter referred to as “the device”) for quantitatively or qualitatively examining and judging the presence of virus infection, such as HIV, or the presence of prion infection is described as an embodiment of the present invention with reference to the drawings.

### [1] VIS-NIR Spectrum Measurement and Data Analytical Methods Using the Device

[1.1] Outline of VIS-NIR Spectrum Measurement

For the examination and diagnosis to be carried out with the device, a method of the present invention is employed. That is, the presence of virus infection, such as HIV, or the presence of prion infection is examined and judged quantitatively or qualitatively by (a) irradiating a sample derived from an examinee or other animal with light having a wavelength in a range of 400 nm to 2500 nm or in part of the range; (b) detecting reflected light, transmitted light, or transmitted and reflected light to obtain absorption spectral data; and (c) analyzing the absorbance at all measurement wavelengths or at a specific wavelength in the absorption spectral data by using an analytical model prepared beforehand.

A first feature point of the device resides in performing viral disease and prion disease diagnoses simply and quickly with high accuracy. This device can also perform an antemortem diagnosis of prion disease, using a blood sample. The wavelength of light with which the sample is irradiated is in the range of 400 nm to 2500 nm or in part of the range (for example, 600 to 1000 nm). This wavelength range can be set as one wavelength region or as a plurality of regions, which include a light wavelength required for examination and judgment to be carried out with the analytical model after the analytical model is prepared.

The light source to be used is, for example, a halogen lamp or an LED, but is not particularly limited. The sample is irradiated with light emitted from the light source directly or through a floodlight means such as a fiber probe. As described later, a pre- or postspectroscopy method can be employed (see FIG. 2). In the prespectroscopy method, the components of light are separated with a spectroscope before the sample is irradiated with it. In the postspectroscopy method, the components of light are separated after the sample is irradiated with it. With respect to the prespectroscopy method, there are two methods, including one of separating components of light emitted from a light source with a prism at the same time, and a method of changing the wavelength continuously by changing the slit space of a diffraction grating. In the latter method, the light emitted from the light source is decomposed at predetermined wavelength intervals, and thereby the sample is irradiated with continuous-wavelength light whose wavelength is changed continuously. In the examples described later, light with a wavelength in the range of 600 to 1000 nm is decomposed at a wavelength resolution of 1 nm, and the sample is irradiated with light whose wavelength is changed continuously in increments of 1 nm.

In regard to the light with which the sample was irradiated, reflected light, transmitted light, or transmitted and reflected light is detected by a detector, and live absorption spectral data can thereby be obtained. Examination and judgment can be carried out with the analytical model by using the live absorption spectral data without further processing. However, preferably, after a data conversion process in which a peak in the spectra obtained above is decomposed into component peaks by a spectroscopic method or multivariate analysis method is carried out, the examination and judgment proceeds with the analytical model by using the converted absorption spectral data. Examples of the spectroscopic method include secondary differential processing and Fourier transformation. On the other hand, examples of the multivariate analysis technique include wavelet conversion and a neural network technique. They are not particularly limited, however.

In the spectral measurement to be carried out with the device, it is preferable that the sample be provided with a perturbation by adding a predetermined condition. This will be described later.

[1.2] Data Analysis Method (preparation of analytical model)

The device examines and diagnoses viral diseases or prion diseases by analyzing the absorbance at a specific wavelength (or at all measurement wavelengths) of the absorption spectral data obtained as described above, with the analytical model. That is, in order to perform final examination and diagnosis, we must prepare the analytical model beforehand. However, this analytical model can also be prepared when the spectra are measured.

In other words, it is desirable that the analytical model be prepared before the measurement. The examination and diagnosis, however, can be carried out by dividing the spectral data obtained at the time of the measurement into two, i.e., data for preparing the analytical model and for examination and diagnosis, and using an analytical model obtained based on the data for preparing it. For instance, when a large quantity of samples is to be examined simultaneously, a part of a sample is used for preparing the analytical model. In this case, the analytical model is therefore prepared at the time of the measurement. In this method, the analytical model can be prepared without requiring teacher data, and this technique is applicable to both the quantitative and qualitative models.

The analytical model can be prepared by the multivariate analysis. For example, when the amount (concentration) of HIV p24 in a sample is to be estimated for the HIV test, a data matrix that stores absorption spectra at all wavelengths obtained by the spectral measurement is decomposed into a score matrix and a loading matrix by singular value decomposition. Then the main component that summarizes the variation in the amount of p24 in the sample is extracted (principal component analysis). This allows an independent component with low colinearity (=high correlation between predictor variables) to be used for the multiple linear regression analysis. A multiple linear regression analysis using the predictor variable as score and the dependent variable as the amount of p24 is then employed. This makes it possible to prepare the analytical model that is used for estimating the amount of HIV p24 from the absorption spectra at all the measurement wavelengths or at a specific wavelength. These operations of series (multivariate analyses) have been established as a principal component regression (PCR) or a partial least squares (PLS) regression method (reference: Multivariate Analysis for Chemists-Introduction to Chemometrics, Yukihiro Ozaki, Akifumi Uda, Toshio Akai; published by Kodansha, 2002). Examples of the regression analysis include a classical least squares (CLS) method and a cross-validation method in addition to the above.

Although the above-mentioned methods are used for preparing a quantitative analytical model, the multivariate analysis can be used for preparing the qualitative analytical model. Examples of the multivariate analysis include a principal component analysis (PCA), a soft independent modeling of class analogy (SIMCA) method, and a k nearest neighbors (KNN) method for class discrimination. In the SIMCA method, the respective main components of a plurality of groups (classes) are analyzed, and the main component model of each class is prepared. Then an unknown sample is compared to the main component model of each class and is assigned to the class of the main component model that it best matches. Moreover, the class discrimination analysis, such as the SIMCA method, can be said to be a method of classifying absorption spectra and regression vectors into respective classes through pattern recognition.

Preparation of the analytical model using a multivariate analysis such as the SIMCA method or PLS method can be carried out by using self-produced software or commercial multivariate analysis software. Furthermore, the creation of software specialized for an intended use allows quick analysis to be carried out.

An analytical model assembled using such multivariate analysis software is stored as a file. The file is retrieved in examining and diagnosing an unknown sample, and quantitative or qualitative examination and diagnosis is then carried out, using the analytical model with respect to the unknown sample. This makes it possible to carry out a simple and quick virus test and prion test. With respect to the analytical model, it is preferable that a plurality of analytical models such as a quantitative model and a qualitative model be stored as files and that the respective models be updated suitably.

Once the analytical model is prepared, the light with a wavelength required for the examination and diagnosis to be carried out using the analytical model is determined. The device can have a simpler configuration by allowing a sample to be irradiated with light with one or a plurality of the wavelength regions determined above.

[1.3] Suitable VIS-NIR Measurement Method and Data Analytical Method by the Device

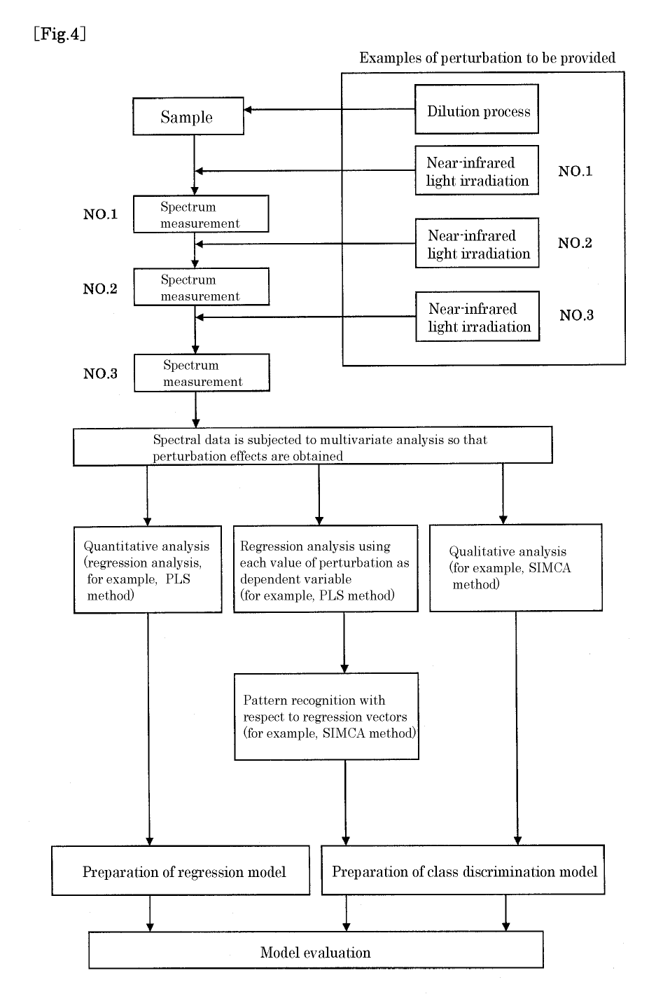

In the spectral measurement by the device, it is preferable that a perturbation be provided for a sample by adding a predetermined condition. Furthermore, in the data analysis by the device, one that brings out an effect of the perturbation is preferred. This is described below with reference to FIG. 4.

[1.3.1] Perturbation

The term “perturbation” denotes that the measurement that is carried out with a plurality of types and conditions being set with respect to a condition causes the change in the absorbance of a sample, and a plurality of different spectral data from each other are obtained. Examples of the condition include any one of the following: change in concentration (including concentration dilution), repeated light irradiation, extension of irradiation time, addition of electromagnetic power, change in light path length, temperature, pH, pressure, mechanical vibration, and one that brings about physical or chemical change by changing the condition, or a combination thereof. The conditions can be classified broadly into (1) those concerning the manner of light irradiation and (2) those concerning the manner of preparation and production of a sample. The conditions (1) and (2) are described below, using examples of repeated light irradiation with respect to the condition (1) and concentration dilution with respect to the condition (2).

As shown in FIG. 4, the repeated light irradiation is carried out by the following method. That is, the spectra of a sample are measured, with a perturbation being added. The perturbation is that a sample is irradiated repeatedly with light continuously or at constant time intervals, and measurement is carried out at a plurality of times. For instance, when a sample is irradiated three times with light consecutively, its absorbance changes slightly (fluctuation), and thereby a plurality of different spectral data from each other is obtained. The use of these spectral data for the multivariate analysis such as the SIMCA method and the PLS method, allows the analytical accuracy to improve, and thus examination and diagnosis also to be carried out with high accuracy. Usually when spectra are measured, a sample is irradiated with light a plurality of times. This is intended to obtain the average value and therefore is different from the “perturbation” that is used herein.

The change in the absorbance of the sample resulting from the perturbation can be considered to be caused by change (fluctuation) in the absorption of water molecules in the sample. That is, it is considered that light irradiation repeated three times, which serves as a perturbation, causes slightly different changes in response and absorption of water at each time, first, second, and third, resulting in fluctuation in spectra.

In the examples described later, a regression analysis was performed by the PLS method using respective absorption spectral data obtained by such irradiation that was repeated three times. Thus the amount of HIV p24 in each sample could be determined well.

Furthermore, in the case of such light irradiation being repeated three times, class discrimination is carried out by the SIMCA method, using at least two of the three absorption spectral data thus obtained. Thus each sample can be classified well, and a highly accurate examination and diagnosis can be performed. The number of light irradiations is not limited to three, but about three times are preferred when, for example, the complications of data analysis are taken into consideration.

On the other hand, in the case of perturbation provided by the concentration dilution, samples that are obtained by diluting a sample to several levels are prepared, and each sample is then subjected to the spectral measurement. This allows a plurality of spectral data to be obtained with respect to one sample, and the use of these spectral data for the multivariate analysis allows a highly accurate examination and diagnosis to be performed. In this case, examples of the multivariate analysis include the following as described later: first, the PLS regression analysis is performed with respect to each sample, with the rate of dilution being used as a dependent variable, and subsequently the regression vectors thus obtained are classified by the use of pattern recognition, for example, by the SIMCA method. Using the class discrimination model thus prepared, the regression vector (pattern) of a class to which the regression vector of an unknown sample is similar is judged and the regression vector of the unknown sample is classified. Thus examination and diagnosis can be performed.

In the examples described later, samples obtained by diluting a sample by a factor of 10 in 10 levels were used, but the number of dilutions and the degrees of dilutions are not particularly limited. These numeral values can be set arbitrarily, since it is enough that the perturbation provided by the concentration dilution can cause fluctuation in spectra obtained thereby.

In the examples described later, the amount of HIV p24 present in a sample at a very low concentration (pg/mL order) could be determined with high accuracy by the PLS method, using a sample that had been diluted 10 times. Therefore it is considered that the method and device of the present invention make it possible to quantify the target substance present in a sample at a very low concentration (pg/mL order). Furthermore, in the case of using a sample whose concentration was diluted about 10⁵ times, respective samples could be classified into infected or noninfected samples by the SIMCA method without misclassification. Thus it is considered that the method and device of the present invention allow class discrimination to be performed even when a target substance is present in the sample at a very low concentration (femto g/mL order). As described above, the present invention makes it possible to carry out examinations with very high accuracy.

Similarly, with respect to conditions of perturbations other than the concentration dilution and repeated light irradiation, a plurality of types and conditions with respect to each condition are set so as to cause fluctuation in the resultant spectra, and the spectral measurement is performed (see Japanese Patent Application No. 2003-379517).

[1.3.2] Method of Data Analysis for Bringing Out Perturbation Effects

The phrase “data analysis for bringing out perturbation effects” denotes that an analytical model is prepared by using a plurality of spectral data obtained with a perturbation with respect to one sample, and a data analysis is carried out by using the analytical model. Specific examples of the data analysis method include the following three methods (see FIG. 4).

(a) Quantitative analysis: a method of determining the amount of a target substance in a sample, such as the amount of HIV p24, by using a quantitative model prepared by a regression analysis such as the PLS method

The quantitative model is prepared by using a plurality of spectral data obtained through the perturbation with respect to one sample. The quantification of the target substance in a sample makes it possible to estimate not only the presence of virus infection and prion infection, but also, for example, the degree of infection, the infection period (such as a window period or an aids period), the degree of seriousness, and the degree of progress.

(b) Qualitative analysis 1: a method of examining and judging the presence of infection using a qualitative model prepared by a class discrimination analysis such as the SIMCA method

The qualitative model is prepared by using a plurality of spectra data obtained through the perturbation with respect to one sample. The preparation of at least three classes of class discrimination models makes it possible to estimate not only the presence of virus infection and prion infection, but also, for example, the degree of infection, the infection period, the degree of seriousness, and the degree of progress.

(c) Qualitative analysis 2: a method of examining and judging the presence of infection by (1) performing a regression analysis (such as the PLS method), using, as a dependent variable, respective values of a perturbation (respective values obtained by varying the condition to provide a perturbation) such as concentration dilution values (the rate of dilution), and (2) using a qualitative model prepared by performing a class discrimination analysis such as the SIMCA method with respect to the regression vectors obtained by the regression analysis.

As described above, the regression analysis is performed using a plurality of spectral data obtained through the perturbation with respect to one sample. It is possible to estimate not only the presence of virus infection and prion infection, but also, for example, the degree of infection, the infection period, the degree of seriousness, and the degree of progress by preparing at least three classes of class discrimination models and carrying out classification through pattern recognition.

Specific Configuration of the Device

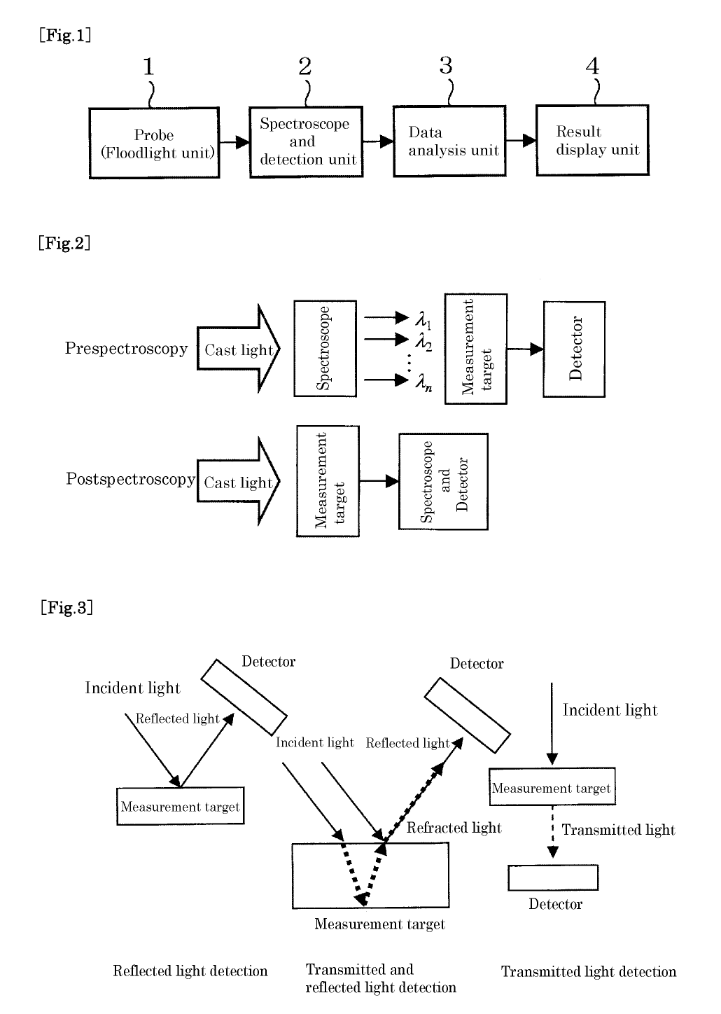

As shown in FIG. 1, an examination and diagnosis system of the device can be configured with four components, a probe (floodlight part) 1, a spectroscope and detection unit 2, a data analysis unit 3, and a result display unit 4. The respective components are described below.

[2.1] Probe (floodlight means)

The probe 1 has a function of guiding light (in the whole wavelength range of 400 nm to 2500 nm or in part of the range) emitted from a light source, such as a halogen lamp or an LED, to a sample, a measurement target. For instance, the probe 1 can be a fiber probe and have a configuration in which light is cast over a measurement target (sample) through a flexible optical fiber. Generally, a probe for a near-infrared spectroscope can be produced inexpensively and thus is low in cost.

The probe 1 can have a configuration in which light emitted from a light source is cast directly over a sample, a measurement target. In that case the probe is not necessary and the light source serves as a floodlight means.

As described above, once an analytical model is prepared, the light wavelength required for the examination and diagnosis to be carried out using the analytical model is determined. The device can have a simplified configuration by employing a configuration in which a sample is irradiated with light in one or more of the wavelength regions determined above.

Furthermore, as described above, it is preferable that the device be provided suitably with a configuration required for providing a perturbation, because it performs the spectral measurement with the perturbation being provided.

[2.2] Spectroscope and Detection Unit (spectroscopic means and detection means)

The device has a configuration of a near-infrared spectroscope as a measurement system. Generally, the near-infrared spectroscope allows a measurement target to be irradiated with light and detects, in a detection unit, reflected light, transmitted light, or transmitted and reflected light with respect to the target. Furthermore, in regard to the light thus detected, the absorbance with respect to incident light is measured at each wavelength.

A spectroscopic system includes pre- and postspectroscopy (see FIG. 2). In prespectroscopy, light is separated into its spectral components before being cast over a measurement target. In postspectroscopy, light from the measurement target is detected and is separated into its spectral components. The spectroscope and detection unit 2 of the device can employ either prespectroscopy or postspectroscopy as a spectroscopic system.

There are three types of detection methods, a reflected light detection, a transmitted light detection, and a transmitted and reflected light detection (see FIG. 3). As shown in FIG. 3, in the reflected light detection and transmitted light detection, light reflected from and light transmitted through a measurement target each are detected by a detector. In the case of the transmitted and reflected light detection, a detector detects light that has entered a measurement target to become a refracted light, which is reflected inside the target and is then again emitted outside the target, which interferes with the reflected light. The spectroscope and detection unit 2 of the device can employ any one of the reflected light detections, transmitted light detections, and transmitted and reflected light detections as a detection system.

The detector in the spectroscope and detection unit 2 can be formed, for example, of a charge coupled device (CCD), which is a semiconductor device, but is not limited to this. Another photodetector can be used for the detector. The spectroscope also can be formed by a known method.

[2.3] Data Analysis Unit (data analysis means)

Absorbance at each wavelength, i.e., absorption spectral data, is obtained from the spectroscope and detection unit 2. The data analysis unit 3 carries out a virus test or a prion test based on the absorption spectral data by using an analytical model prepared beforehand as described above.

In regard to the analytical model, it also is possible to prepare a plurality of analytical models, including a quantitative model and a qualitative model, and to use a suitable one according to the type of evaluation, to be carried out, i.e., a quantitative evaluation or a qualitative evaluation. Moreover, with respect to the analytical models, when both one for a virus test and one for a prion test are prepared beforehand, they can make it possible to carry out both the tests in one device, or when different types of analytical models are prepared according to the type of virus to be examined, they can make it possible to carry out virus tests of a plurality of types in one device.

Data analysis unit 3 can be formed of: a storage unit for storing various data, such as spectral data, programs for a multivariate analysis, and analytical models, and an arithmetic unit that carries out arithmetic processing based on these data and programs. Data analysis unit 3 can be formed, for example, of an IC chip. Therefore the device also is easily reduced in size so as to be a portable type. The above-mentioned analytical models also are written in the storage unit, such as an IC chip.

[2.4] Result Display Unit (display means)

Result display unit 4 displays analytical results obtained in the data analysis unit 3. Specifically, it displays the concentration value of a target substance, such as the amount of HIV p24, in a sample obtained as a result of the analysis carried out with analytical models. In the case of a qualitative model, it displays, for example, “infected”, “high possibility of infection”, “low possibility of infection”, “noninfected”, “window period”, and “aids period”, according to the class discrimination results. In the case where the device is of a portable type, the result display unit 4 is preferably a flat display formed, for example, of liquid crystal.

### [3] Applications of the Device

The device can be any one of the following: (1) one for a virus test, (2) one for a prion test, and (3) one for both virus and prion tests. The device can be configured as one for a test of a specific virus, such as HIV, (special purpose) or as one for a test of a plurality of types of viruses (general purpose).

The virus to be examined is not particularly limited. Besides HIV, examples thereof include hepatitis viruses such as hepatitis C virus and hepatitis B virus, as well as various other viruses that cause human or animal viral diseases, such as Boma disease virus (BDV), SARS coronavirus, adult T-cell leukemia virus, human Parvovirus, enterovirus, adenovirus, Coxsackie A and B viruses, echovirus, herpes simplex virus, influenza virus, norovirus, rotavirus, poliovirus, measles virus, and rubella virus.

The present device is used preferably for examination and diagnosis related to these viral diseases. However, the method and device of the present invention are not limited thereto and can be used, for example, for a virus test with respect to food and drink to be carried out as a safety inspection for food and drink.

The prion test can be used for the examination and diagnosis of human or animal prion diseases such as not only for Creutzfeld-Jakob disease (CJD), but also for bovine spongiform encephalopathy (BSE; mad cow disease), which is a prion disease of bovine; Scrapie, a prion disease of sheep and goat; and chronic wasting disease, a prion disease of deer.

### EXAMPLES

Examples of the present invention are described below, but do not limit the present invention.

### Example 1

### Examination and diagnosis of the presence of HIV infection by near-infrared spectroscopy

[1.1] Measurement of Absorption Spectrum

In this example, the absorption spectra of each sample were measured by the following measurement method.

With respect to a total of 13 specimens, including 5 of normal donor plasma and 8 of HIV-infected plasma, their concentrations each were gradually diluted with a PBS buffer solution up to 10 levels, and the 10-times diluted series (the concentration is diluted from 10⁻¹ times to 10⁻¹⁰ times by a factor of 10) thus obtained were used as samples.

Two mL of each sample was placed in a polystyrene cuvette and measurement was then carried out, with a perturbation of repeated light irradiation being provided using a near-infrared spectroscopic system (trade name: Fruit-Tester-20 [FT-20], Japan Fantec Research Institute, Shizuoka Japan). Specifically, the sample was irradiated three times consecutively with light having a wavelength of 600 to 1000 nm, and absorption spectra were measured by detecting each reflected light. The wavelength resolution was 1 nm. The length of the optical path that passes through the sample was set at 10 mm.

[1.2] Analysis of absorption spectrum

In this example, the resultant absorption spectra were subjected to various analyses, and studies were made with respect to the optimal analytical model.

[1.2.1] First analysis method: Classification by SIMCA method and HIV diagnosis First, the resultant absorption spectra were analyzed at each rate of dilution by the SIMCA method. An analytical example of a sample diluted 10 times is described below.

In order to prepare analytical models, the amount of HIV p24 antigens in each sample (diluted 10 times) was measured by the ELISA method. Furthermore, the presence of HIV genes was examined by the PCR method, and the presence of anti-HIV antibodies was also examined. The results are indicated in Table 1 below.

TABLE 1

| | Sample | HIV p24 | HIV | Anti-HIV | |
|---|---|---|---|---|---|
| | name | [pg/ml] | PCR | ½ | Class |
| Normal donor plasma | No 1-1 | 0 | NT | (−) | 1 |
| Normal donor plasma | No 2-1 | 0 | NT | (−) | 1 |
| Normal donor plasma | No 3-1 | 0 | NT | (−) | 1 |
| Normal donor plasma | No 4-1 | 0 | NT | (−) | 1 |
| Normal donor plasma | No 5-1 | 0 | NT | (−) | 1 |
| HIV-infected plasma | No 6-1 | 0.44 | (+) | (−) | 3 |
| HIV-infected plasma | No 7-1 | 8.31 | (+) | (−) | 2 |
| HIV-infected plasma | No 8-1 | 0.18 | (+) | (−) | 3 |
| HIV-infected plasma | No 9-1 | 22.93 | (+) | (−) | 2 |
| HIV-infected plasma | No 10-1 | 21.47 | (+) | (−) | 2 |
| HIV-infected plasma | No 11-1 | 0.00 | (+) | (−) | 3 |
| HIV-infected plasma | No 12-1 | 0.28 | (+) | (−) | 3 |
| HIV-infected plasma | No 13-1 | 36.48 | (+) | (−) | 2 |

* (+): positive; (−): negative; and NT: not tested

In Table 1, “HIV p24” denotes the measurement results of the amount of p24 antigens; “HIV PCR” denotes the result of examination of the presence of HIV genes; “Anti-HIV 1/2” denotes the results of an examination of the presence of anti-HIV antibodies; (+) means positive; (−) means negative; and “NT” denotes not tested. With respect to “HIV p24”, the detected values smaller than 1 pg/mL are indicated as negative (−). Based on these results, the 13 specimens were divided into three classes, 1 to 3, according to the following classifications:

Class 1: HIV p24 (−), HIV PCR (−), Anti-HIV (−) (HIV noninfected);

Class 2: HIV p24 (+), HIV PCR (+), Anti-HIV (−); and

Class 3: HIV p24 (−), HIV PCR (+), Anti-HIV (−).

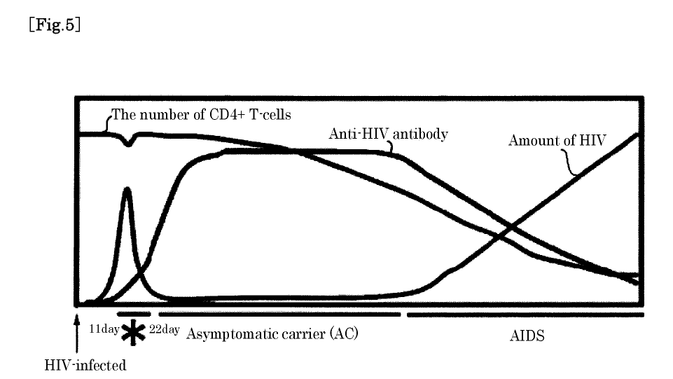

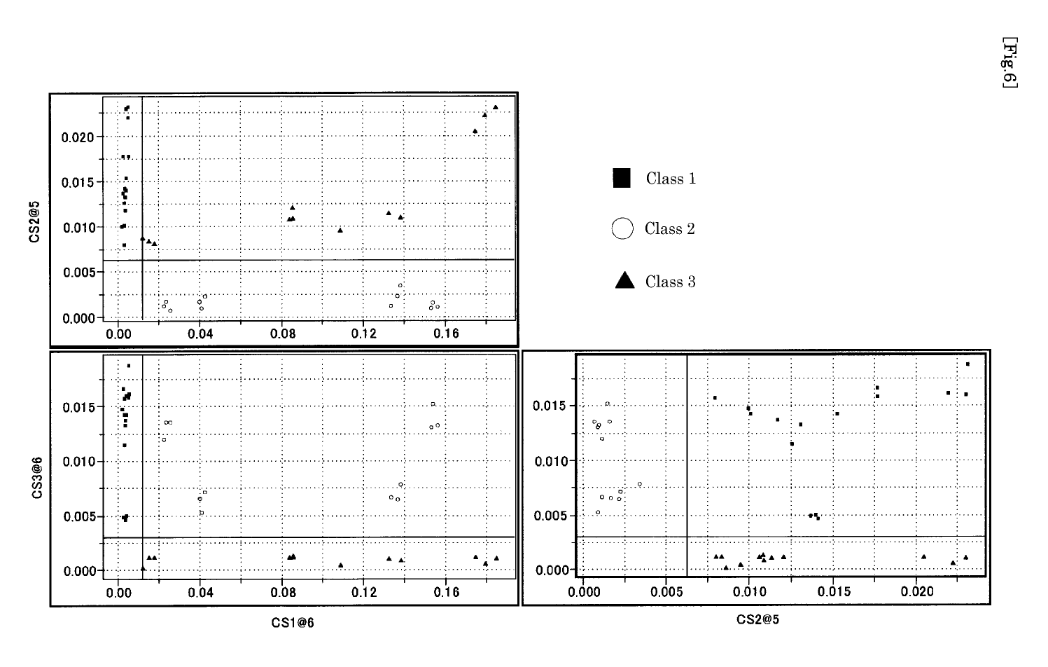

The amount of HIV antigens in the blood of a patient after HIV infection, the number of CD4 positive T-cells, and the amount of anti-HIV antibodies change as shown in FIG. 5. Each of the specimens of Classes 2 and 3 described above belongs to a window period (11 days after infection or more) with a large amount of HIVs shown with an asterisk (*) in FIG. 5. However, Class 2 includes a sample of the period in which p24 can be detected by the ELISA method, and Class 3 includes a sample of the period in which HIVs exist, but p24 cannot be detected by the same method. That is, Class 3 is a very early stage (corresponding to a period in which the amount of viruses has not increased so much after infection) in the window period. It is a specimen group that can be detected by only a highly sensitive method called PCR.

In this example, a commercial software for a multivariate analysis (Pirouette ver.3.01 (trade name), Informetrics) was used for preparing an analytical model, and the SIMCA analysis was carried out with the algorithm described below:
Number of included samples: 39
Preprocessing: Autoscale
Scope: Local
Maximum factors: 11
Optimal factors: 6,5,6
Probability threshold: 0.9500
Calibration transfer: Not enabled
Transform: Smooth (25)

Brief descriptions of the above-mentioned algorithms follow. The “number of included samples” denotes the number of samples used for analysis (the number of spectra). The number of samples, 39, denotes that three absorption data obtained through three-time consecutive irradiations, respectively, were used per sample that had been diluted 10 times. “Preprocessing” denotes a preliminary treatment, and “Autoscale” denotes the use of a method in which the average is determined after distributed scaling. For “Scope”, there are global and local types. In this case, the local type was selected. The item “maximum factors” denotes the number of factors (main component) to be analyzed to the maximum, and the selectable maximum number of factors was selected. The term “optimal factors” indicates the number of factors that were most suitable for preparing analytical result models, and “6,5,6” denotes that the most suitable number of factors in Class 1 is up to 6, the most suitable number of factors in Class 2 is up to 5, and the most suitable number of factors in Class 3 is up to 6. “Probability threshold” means the threshold in judging whether it belongs to a certain class. “Calibration transfer” denotes whether mathematical adjustment is carried out for reducing the difference between devices. “Transform” means conversion, and “smooth” denotes that smoothing was carried out. The conversion of smoothing denotes that based on the principle of a Savitzky-Golay polynomial filter, convolution to the predictor variable in a window, including a point of central data and n points on one side, was carried out, and n=25 was selected.

FIG. 6 shows a Coomans plot obtained as a result of the SIMCA analysis. Table 2 indicates the results of interclass distances, and Table 3 indicates the results of misclassification.

TABLE 2

| | CS1@6 | CS2@5 | CS3@6 |
|---|---|---|---|
| CS1 | 0.0000 | 17.5441 | 21.5177 |
| CS2 | 17.5441 | 0.0000 | 5.2285 |
| CS3 | 21.5177 | 5.2285 | 0.0000 |

In Table 2, CS1, CS2, and CS3 denote Class 1, Class 2, and Class 3, respectively (hereinafter, the same applies). Furthermore, CS1@6 means that 6 factors (main components) are used in Class 1. The same applies below, and the number described after “@” denotes the number of factors that were used. When the interclass distance is 3 or more, it can be considered that the interclass discrimination has been achieved.

TABLE 3

| | Pred1@6 | Pred2@5 | Pred3@6 | No match |
|---|---|---|---|---|
| Actual 1 | 15.0000 | 0.0000 | 0.0000 | 0.0000 |
| Actual 2 | 0.0000 | 12.0000 | 0.0000 | 0.0000 |
| Actual 3 | 0.0000 | 0.0000 | 12.0000 | 0.0000 |

In Table 3, “Actual 1” denotes that the actual class is “1”, and the same applies to the others. Furthermore, “Pred1” means that the class estimated using an analytical model obtained by the SIMCA analysis is “1”, and the same applies to the others. “No match” denotes the mismatch between the actual class and the estimated class is indicated by numerical values. In the case of “0”, it means they are matched completely. Moreover, the numerical number, for example, “15” in the table denotes that since 5 samples of Class 1 were measured three times, 5×3=15 spectra were analyzed. All 15 spectra were judged correctly as Class 1.

From the results described above, the respective samples were classified well by using the analytical model obtained by the SIMCA analysis. The analytical model thus assembled is stored as a file, which is retrieved when an unknown sample is to be examined and diagnosed, and the class into which the unknown sample is classified is estimated by using the analytical model. This allows HIV infection to be examined and diagnosed simply and quickly.

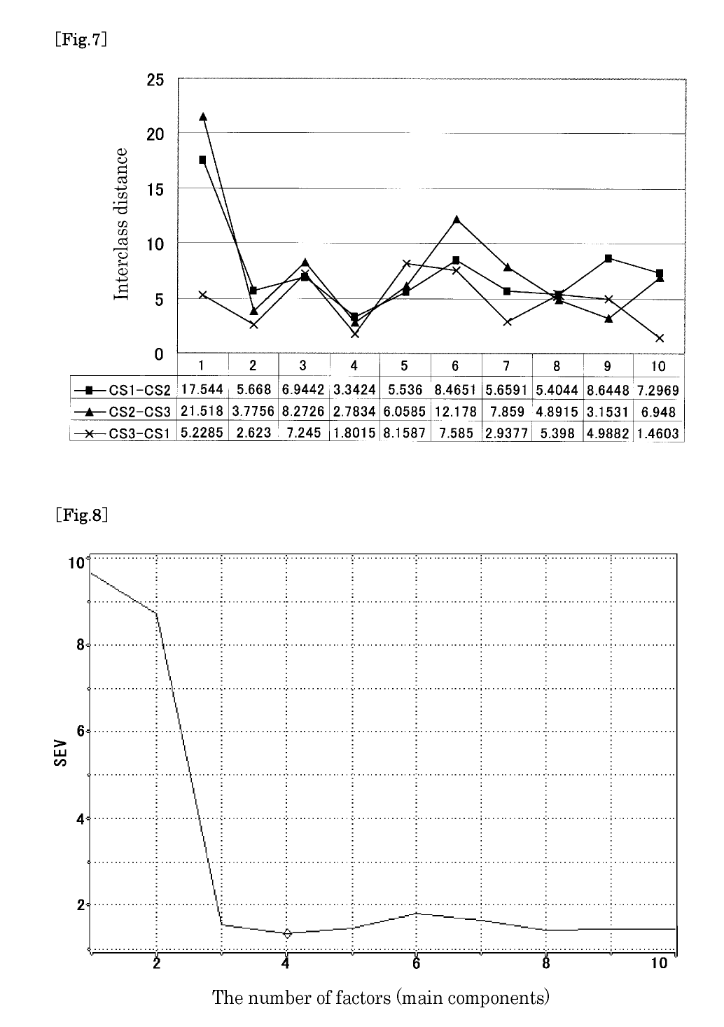

In the above, an analysis example of a sample that had been diluted 10 times was described. The samples that had been diluted 100 times or more also were analyzed in the same manner. FIG. 7 is a graph showing the results indicated with respect to the interclass distance. In FIG. 7, “1” to “10” indicate the results of the analyses of samples that have been diluted 10¹ to 10¹⁰ times. Even in the case of a sample that had been diluted 10¹⁰ times, it was possible to carry out the interclass discrimination by using the resultant analytical model. Thus it is considered that this method has higher accuracy as compared to conventional HIV examination and diagnosis, and therefore the examination and diagnosis can be carried out by using a further trace amount of sample. Moreover, when, for instance, the result of misclassification is taken into consideration, an analytical model with relatively high accuracy was prepared in the case of samples that had been diluted 10¹ to 10⁵ times (in the case of concentrations of 10⁻¹ to 10⁻⁵ times).

[1.2.2] Second analysis method: estimation of the amount of HIV p24 by the PLS method and HIV diagnosis

Next, a regression analysis was carried out by the PLS method, and a quantitative model of the amount of HIV p24 antigens was prepared. The samples used herein were four (Samples 7, 9, 10, and 13) that belonged to Class 2 in which HIV p24 was (+), positive. Each sample used herein was one diluted 10 times.

With the numeral value of dependent variable being the amount of HIV p24 (pg/mL), the PLS regression analysis was carried out with the algorithm described below:
Number of included samples: 12
Preprocessing: Autoscale
Maximum factors: 10
Optimal factors: 3
Validation: Step (1)
Probability threshold: 0.9500
Calibration transfer: Not enabled
Transform: Smooth (25)

The number of samples, 12, denotes that three absorption data obtained through three-time consecutive irradiations, respectively, were used with respect to the aforementioned four samples that had been diluted 10 times. The respective items denote the same as described above. For “validation”, there are step and cross validations. The number described in the brackets indicates the number of samples to be removed.

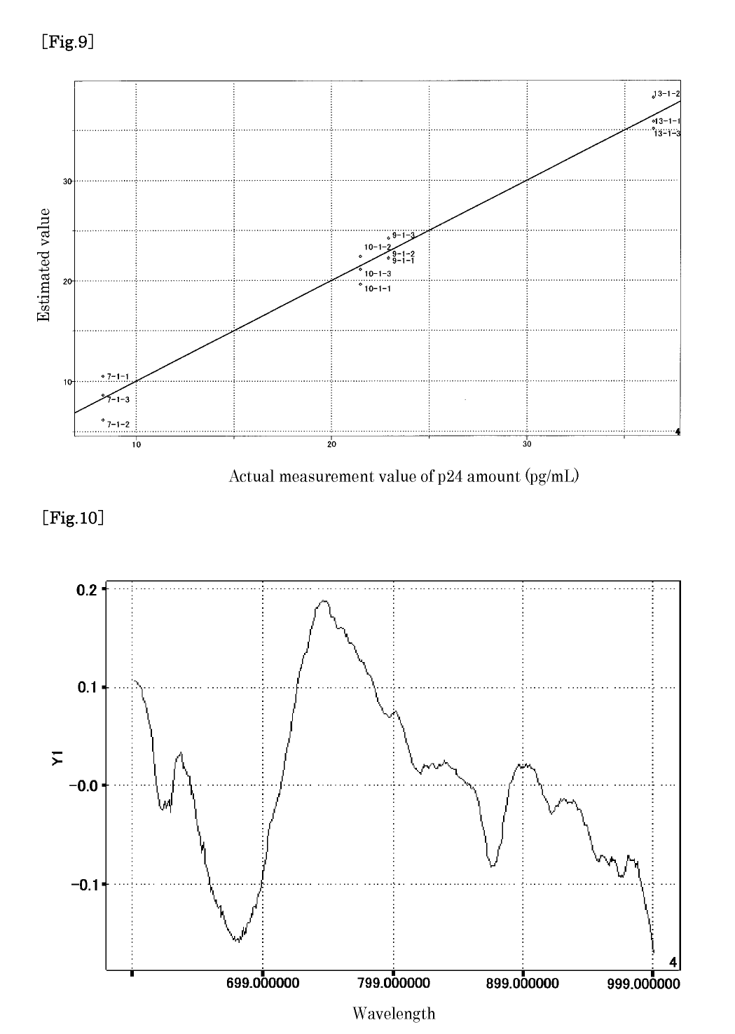

FIG. 8 shows the results of factor selections. In FIG. 8, the horizontal axis indicates the factor number that was used, and the vertical axis indicates the standard error of cross-validation (SEV). In this factor selections, a factor number 4 (in this case, the correlation coefficient r is 0.9908) that allows the SEV to be minimum was selected, and the PLS regression was carried out. FIGS. 9 and 10 show the analytical results thereof. In FIG. 9, the X axis indicates the numerical value of dependent variable (i.e., the amount of HIV p24 [pg/mL]), and values estimated with a quantitative model obtained by the analysis with respect to the numerical values are plotted on the Y axis. In FIG. 9, for example, “10-1-3” denotes data of the third irradiation with respect to a sample 10 that has been diluted 10¹ times with PBS (the same applies below). FIG. 10 shows all the partial regression coefficients (regression vectors) of a multiple regression type prepared as a quantitative model. The horizontal axis indicates a wavelength, and the vertical axis indicates a coefficient value. The wavelength employed herein was 600 nm to 1000 nm, and the wavelength resolution was 1 nm.

As shown in FIG. 9, the amount of HIV p24 [pg/mL] of each sample was estimated with high accuracy by using the analytical model obtained by the PLS analysis. The analytical model thus assembled is stored as a file, which is retrieved when an unknown sample is to be examined and diagnosed, and the amount of HIV p24 [pg/mL] of the unknown sample is estimated by using the analytical model. This allows HIV infection to be examined and diagnosed simply and quickly.

As a result of the analysis, it was found that eight wavelengths, 686 nm, 731 nm, 755 nm, 802 nm, 879 nm, 918 nm, 954 nm, and 979 nm, considerably contributed to HIV quantification. The specific values of these wavelengths can be changed depending on, for example, the measurement conditions or solvent. Moreover, HIV quantification can also be performed by using a quantitative model prepared with wavelengths other than those described above.

[1.2.3] Third analysis method: pattern recognition with regard to the regression vector obtained by the PLS regression analysis, and HIV diagnosis

Next, using all samples that had been diluted 10¹ to 10¹⁰ times per sample, the PLS regression analysis was performed with the algorithm described below:
Number of included samples: 30
Preprocessing: Autoscale
Maximum factors: 28
Optimal factors: 5
Validation: Step (1)
Probability threshold: 0.9500
Calibration transfer: Not enabled
Transform: Log10 Smooth (25)

The number of samples, 30, denotes that three absorption data obtained through three-time consecutive irradiations, respectively, were used with respect to each of the samples that had been diluted 10¹ to 10¹⁰ times. The respective items denote as described above. “Log10” in the item “transform” denotes that each predictor variable was converted with common logarithm. In other words, in this analysis the dependent variable was defined as log₁₀(10¹)=1 in the case of the sample diluted 10¹ times. In the same manner, it was defined as log₁₀(10²)=2 in the case of the sample diluted 10² times and as log₁₀(10³)=3 in the case of the sample diluted 10³ times, for example.

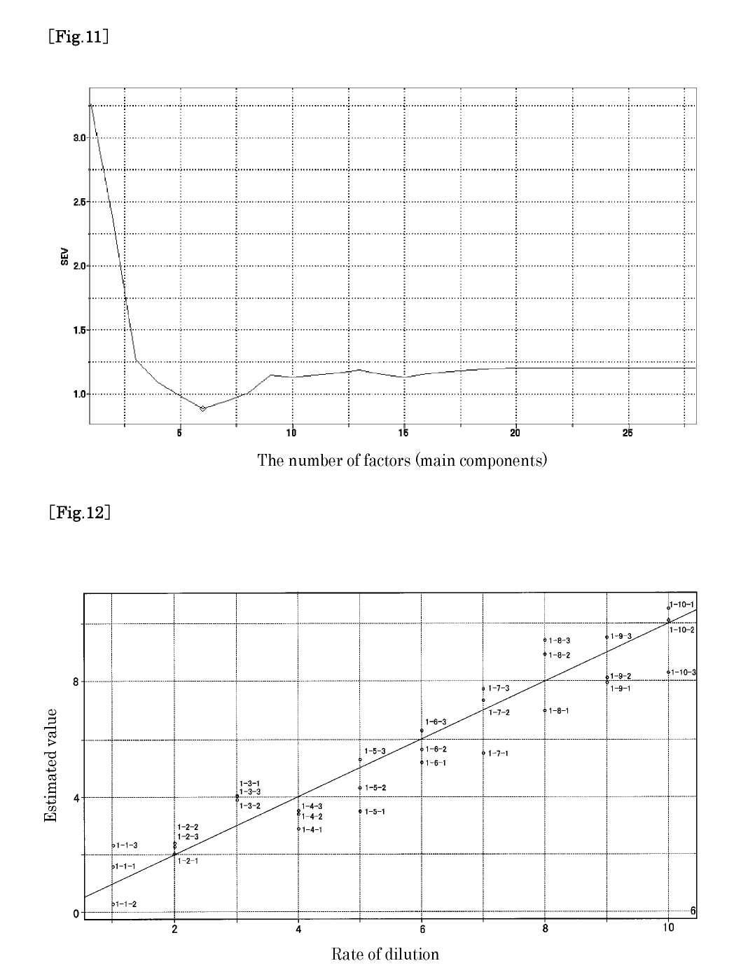

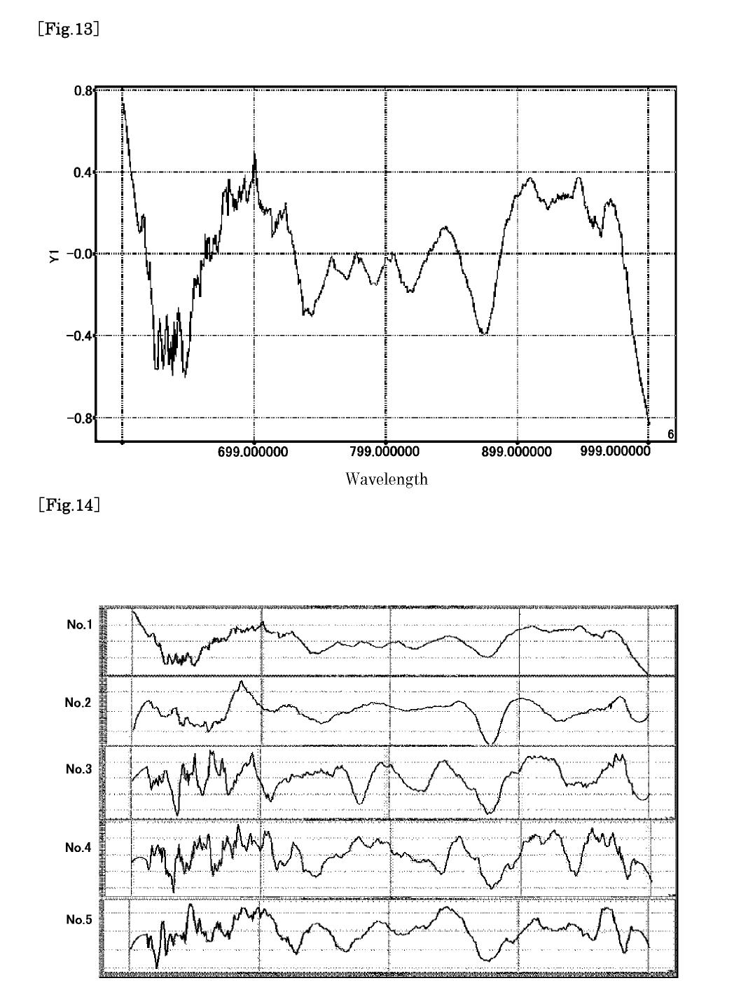

FIG. 11 shows the results of factor selections performed with respect to Sample 1. In this figure, the horizontal axis indicates the factor number that was used, and the vertical axis indicates the standard error of cross-validation (SEV). In this factor selections, a factor number 6 (in this case, the correlation coefficient r is 0.9520) that allows the SEV to be minimum was selected, and the PLS regression was carried out. FIGS. 12 and 13 show the analytical results thereof. In FIG. 12, the X axis indicates the numerical value of dependent variable (i.e., the rate of dilution), and values estimated with a quantitative model obtained by the analysis with respect to the numerical values are plotted on the Y axis. FIG. 13 shows all the partial regression coefficients (regression vectors) of a multiple regression type prepared as a quantitative model. The horizontal axis indicates a wavelength, and the vertical axis indicates a coefficient value. The wavelength employed herein was 600 nm to 1000 nm, and the wavelength resolution was 1 nm.

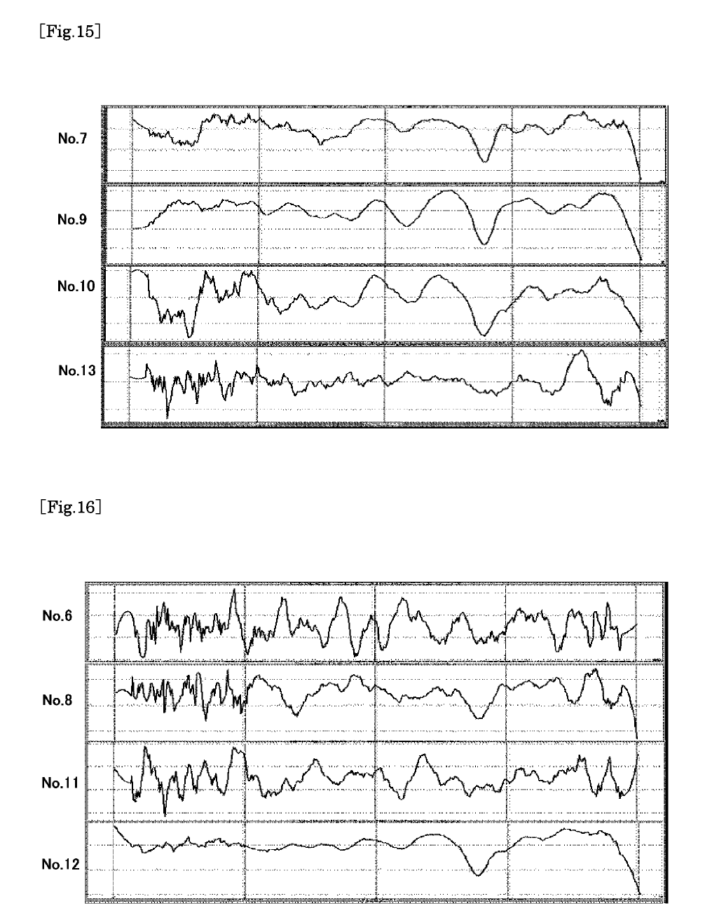

The same analysis as described above was carried out in regard to Samples 2 to 13. In FIGS. 14 to 16, the regression vectors of the respective samples thus obtained are shown by comparison with respect to each class. FIG. 14 shows, by comparison, the respective regression vectors of Samples 1 to 5 that belong to Class 1. By comparison, FIG. 15 shows the respective regression vectors of Samples 7, 9, 10, and 13 that belong to Class 2. By comparison, FIG. 16 shows the respective regression vectors of Samples 6, 8, 11, and 12 that belong to Class 3. The regression vectors obtained by such analyses are stored as a reference database, and the presence of HIV infection can be examined and diagnosed by comparing the regression vector of an unknown sample with stored regression vectors to examine which of the regression vectors of a normal donor sample and that of an HIV infected sample is close to that of the unknown sample by using pattern recognition, for instance, of the SIMCA method.

With respect to Samples 1 to 13, the regression vectors obtained by the aforementioned analyses are taken as spectra, and the SIMCA analysis was really carried out with the same algorithm as in the first analysis method. As a result, these samples were classified well. Table 4 indicates the results of interclass distances, and Table 5 indicates the results of misclassification.

TABLE 4

| | CS1@3 | CS2@2 | CS3@2 |
|---|---|---|---|
| CS1 | 0.0000 | 2.4620 | 2.7731 |
| CS2 | 2.4620 | 0.0000 | 3.8988 |
| CS3 | 2.7731 | 3.8988 | 0.0000 |

TABLE 5

| | Pred1@3 | Pred2@2 | Pred3@2 | No match |
|---|---|---|---|---|
| Actual 1 | 5.0000 | 0.0000 | 0.0000 | 0.0000 |
| Actual 2 | 0.0000 | 4.0000 | 0.0000 | 0.0000 |
| Actual 3 | 0.0000 | 0.0000 | 4.0000 | 0.0000 |

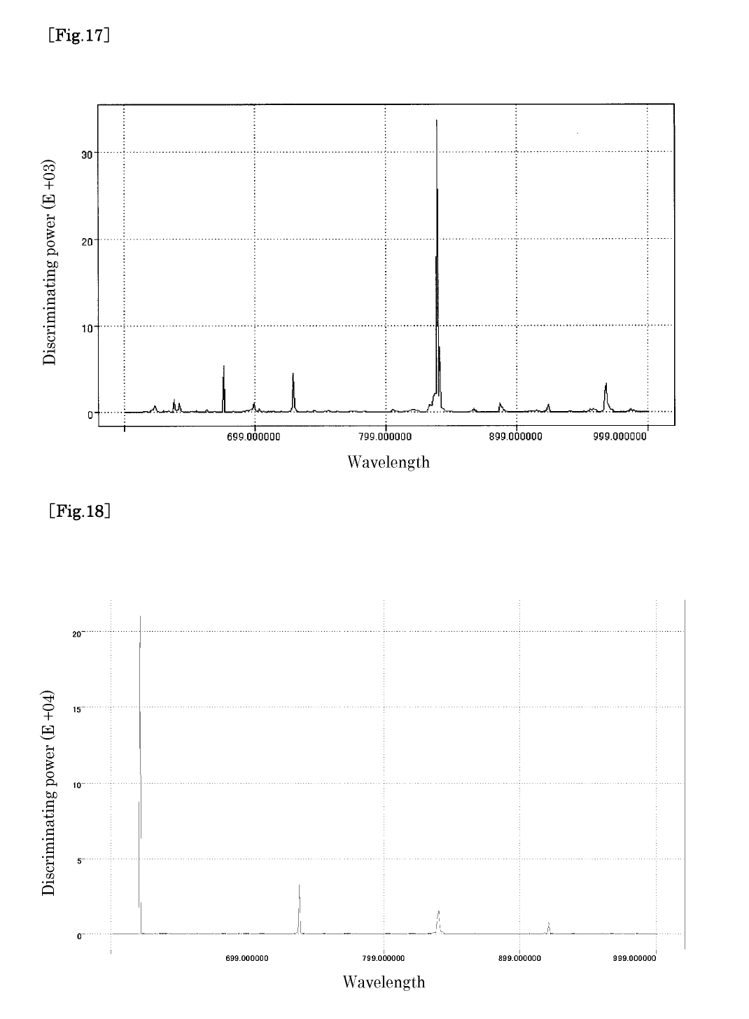

FIG. 17 shows discriminating power (vertical axis) at each wavelength (horizontal axis) obtained as a result of the SIMCA analysis. FIG. 17 shows that at the wavelengths at which the value of the discriminating power is higher, the difference in the above-mentioned wavelengths of the three classes from one another increases. That is, it is considered that the sharp peak wavelength at which the discriminating power is high is one of the effective wavelengths for discriminating between normal donor blood plasma and HIV infected blood plasma. Accordingly, the presence of HIV infection can be diagnosed simply and quickly with high accuracy by carrying out discrimination, with attention being focused on the wavelengths obtained by the SIMCA analysis, as described above.

Furthermore, among Samples 1 to 13, all except for Samples 12 and 13 having a heterogeneous pattern were subjected to the SIMCA analysis in the same manner as above. As a result, the respective samples were classified well. In this analysis, the distance between Classes 1 and 2 increased as compared to that obtained by the above-mentioned analysis. Table 6 indicates the results of interclass distances, and Table 7 indicates the results of misclassification.

TABLE 6

| | CS1@2 | CS2@1 | CS3@1 |
|---|---|---|---|
| CS1 | 0.0000 | 14.1603 | 2.2257 |
| CS2 | 14.1603 | 0.0000 | 24.3445 |
| CS3 | 2.2257 | 24.3445 | 0.0000 |

TABLE 7

| | Pred1@2 | Pred2@1 | Pred3@1 | No match |
|---|---|---|---|---|
| Actual 1 | 5.0000 | 0.0000 | 0.0000 | 0.0000 |
| Actual 2 | 0.0000 | 3.0000 | 0.0000 | 0.0000 |
| Actual 3 | 0.0000 | 0.0000 | 3.0000 | 0.0000 |

FIG. 18 shows discriminating power (vertical axis) at each wavelength (horizontal axis) obtained as a result of the aforementioned SIMCA analysis carried out with respect to the samples, except for Samples 12 and 13. It is considered that the sharp peak wavelength at which the discriminating power is high is one of the effective wavelengths for discriminating between normal donor blood plasma and HIV infected blood plasma. Thus the presence of HIV infection can be diagnosed simply and quickly with high accuracy by carrying out discrimination, with attention being focused on the wavelengths obtained by the SIMCA analysis as described above.

[1.2.4] Fourth analysis method: classification by the SIMCA method and HIV diagnosis (experiment for examining the effect of perturbation provided by irradiation repeated three times)

Next, using one of the three absorption data obtained by three-time consecutive irradiations, respectively, data obtained in combination of two of them, or all the three data, the SIMCA analysis was carried out with the same algorithm as in the first analysis method. For the analysis, respective samples diluted 10 times were used.

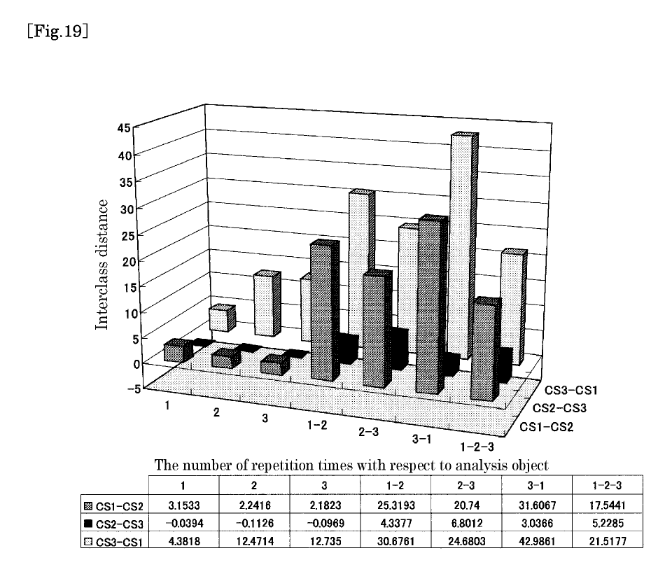

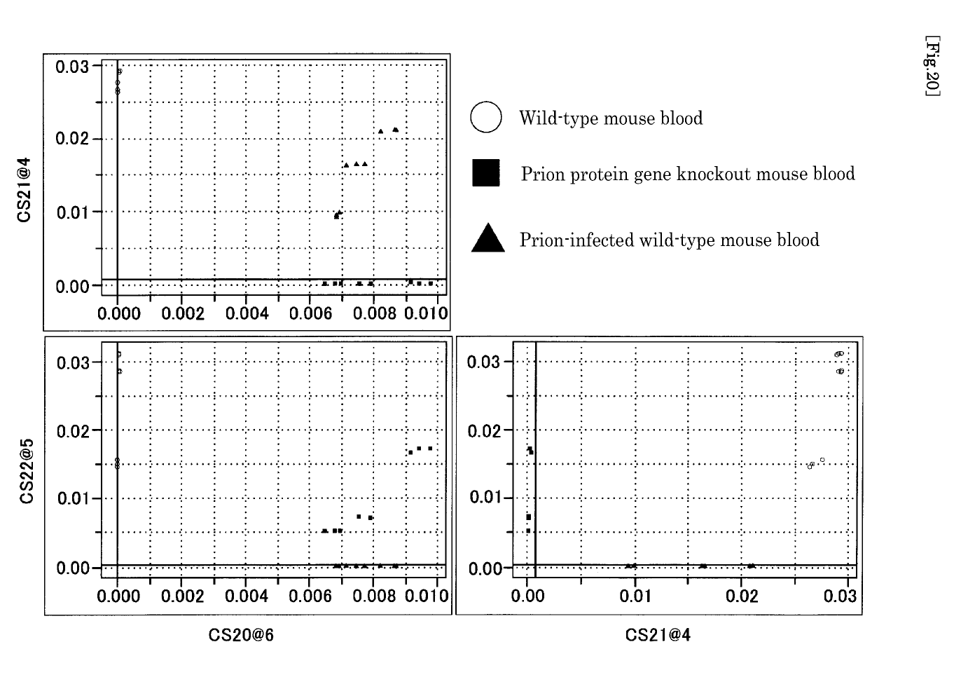

FIG. 19 is a graph showing, with respect to the interclass distance, the results of the above-mentioned analysis. In FIG. 9, numeral “1” denotes the case of using only the data obtained by the first irradiation; numerals “2” and “3” denote the cases of using only the data obtained by the second and third irradiations, respectively; “1-2” denotes the case of using the data obtained by the first and second irradiations; “2-3” denotes the case of using the data obtained by the second and third irradiations; “3-1” denotes the case of using the data obtained by the first and third irradiations; and “1-2-3” denotes the case of using all data obtained by the first to third irradiations.

When, for instance, the results of the interclass distance and the results of misclassification are taken into consideration together, it is considered that excellent examination and judgment can be made when the multivariate analysis is carried out using at least two absorption data selected from the three absorption data obtained by three-time consecutive irradiations. In particular, it is considered that in the case of using at least two absorption data, excellent examination and judgment can be made by carrying out an analysis by using at least two absorption data that include the data obtained by the third irradiation.

### Example 2

### Examination and diagnosis of the presence of prion infection by near-infrared spectroscopy

### [2.1] Measurement of absorption spectrum

In this example, the absorption spectrum of each sample was measured by the following measurement method.

The samples used were brain tissues, brain homogenates, and the blood of a wild-type mouse (WT mouse), a prion protein gene knockout mouse (Rikn PrP −/−mouse), and a prion-infected wild-type mouse (prion-infected WT mouse). The prion was an Obihiro strain and was derived from scrapie. The blood used was obtained by dissolving 10 μL of collected blood in 1 mL of PBS. The brain tissue used as a sample was the tissue from half of a brain. Furthermore, the brain homogenate used was obtained by further dissolving 10% brain homogenate, prepared by being dissolved in 20 μL of LPBS, in 1 mL of LPBS.

The measurement of the absorption spectra was carried out using a near-infrared spectroscopic system in the same manner as in Example 1, except for the preparation of the samples.

[2.2] Analysis of Absorption Spectra

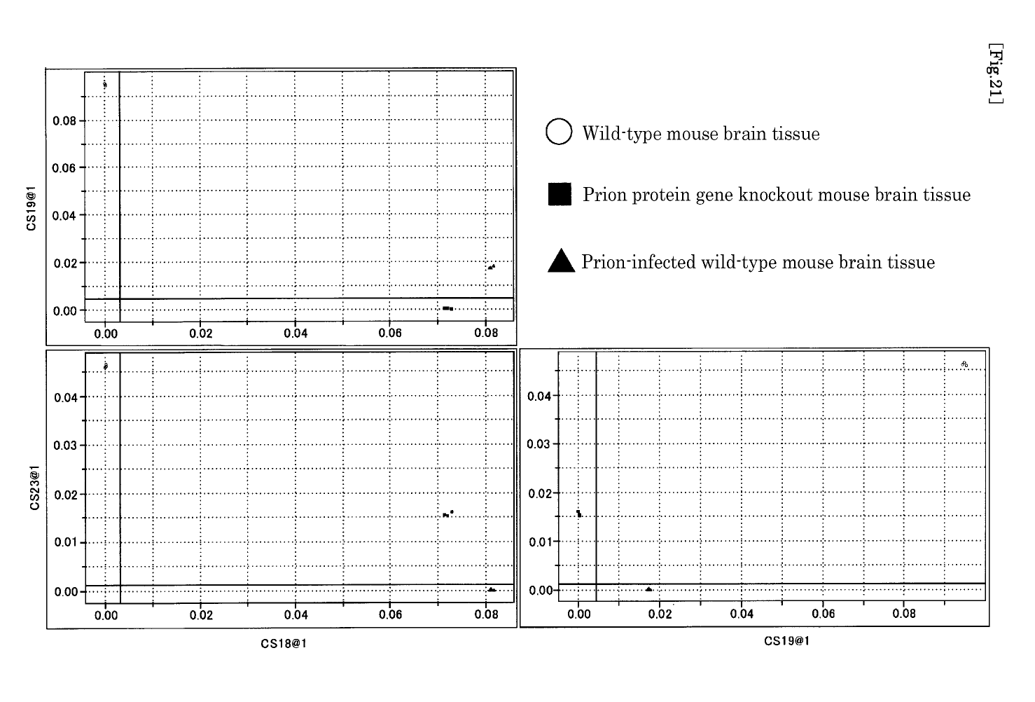

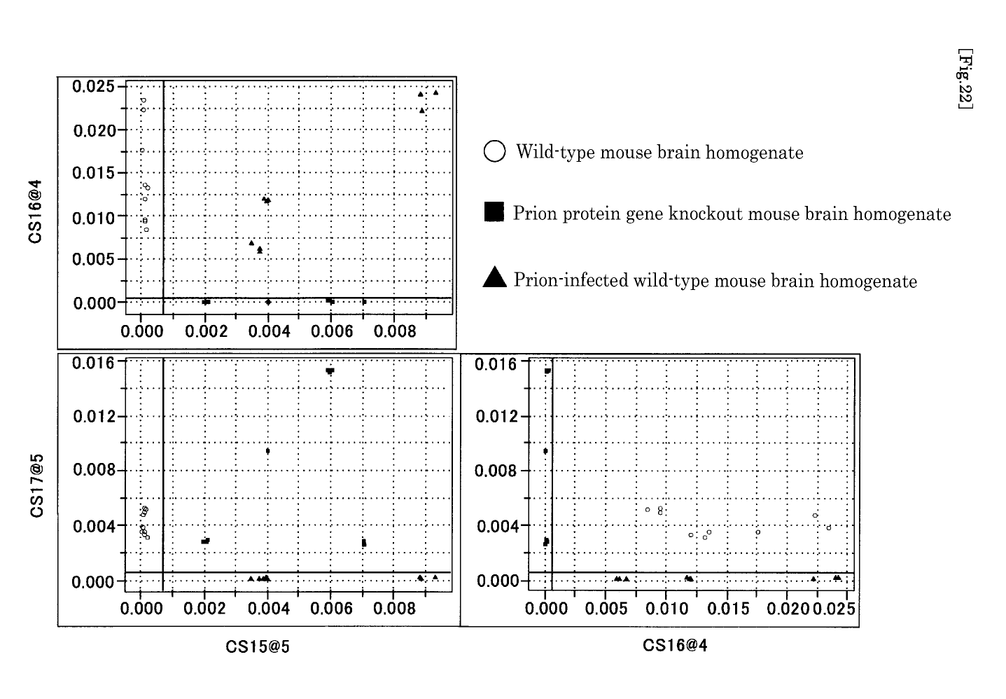

With respect to the resultant absorption spectra, the SIMCA analysis was carried out with the same algorithm as in the first analysis method. FIGS. 20 to 22 each show a Coomans plot obtained as the result of the SIMCA analysis. FIG. 20 shows the results obtained using the blood samples. FIGS. 21 and 22 show the results obtained using brain tissues and brain homogenate for the samples, respectively.

As shown in these figures, according to the analysis, the samples of the wild-type mouse were assigned to classes (CS) 15, 18, and 20, those of the knockout mouse were assigned to classes (CS) 16, 19, and 21, and those of the prion-infected mouse were assigned to classes (CS) 17, 23, and 22.

As described above, similarly in the case of using any one of the blood, brain tissue, and brain homogenate for a sample, the respective samples of the prion-infected animal, prion-noninfected animal, and prion knockout animal were classified well by using the analytical model obtained by the SIMCA analysis. Therefore the analytical model thus assembled is stored as a file, which is retrieved when an unknown sample is to be examined and diagnosed, and the class into which the unknown sample is classified is estimated by using the analytical model. This allows prion infection to be examined and diagnosed simply and quickly.

Since this method can employ blood as a sample, it is also applicable to an antemortem diagnosis. Furthermore, a large amount of samples can be analyzed with high accuracy by making this measurement online.

Urine, another biological fluid, a tissue, and a tissue extract are also considered to be used as samples, in addition to blood. Furthermore, as described below, it is also possible to carry out a measurement by using a biological part, such as an ear, an abdomen, or a fingertip of a hand or foot, as a specimen without damaging the biological body.

[2.3] Measurement from Ear and Abdomen

Next, studies were made with respect to the possibility of the method of antemortem diagnosis of prion disease, using the near-infrared spectroscopy through measurements from biological ears and abdomens.

The prion-infected mice used for the experiment were C57BL6 mice subjected to intracerebral inoculation of Chandler-strain scrapie-infected brain homogenate and C57BL6 mice subjected to intracerebral inoculation of Obihiro-strain scrapie-infected brain homogenate. The controls used herein were mice subjected to intracerebral inoculation of normal brain homogenate and mice subjected to intracerebral inoculation of PBS. With respect to these mice, the development of prion disease was observed through various symptoms taken as indices, such as shaking, abnormality in step, or whether the animal could get up from the state of lying facing upward. Among the Chandler-strain scrapie-inoculated mice, one developing prion disease was observed about 170 days after inoculation, and all had developed prion disease after about 180 days. On the other hand, among the Obihiro-strain scrapie-inoculated mice, one developing prion disease was observed about 200 days after inoculation, and all had developed prion disease after about 210 days. On the contrary, however, among the mice inoculated with normal brain homogenate and those inoculated with PBS, none had developed prion disease.

The near-infrared spectrometry was carried out over time with respect to the ears of the above-mentioned four types of mice. A fiber probe was used for the spectrometry. The spectrometry was carried out in such a manner that the brain was interposed between an optical output unit and a photo detection unit that were placed against the right ear and left ear, respectively. The other measurement conditions such as the wavelength range that were used for the spectrometry were the same as in Example 1.

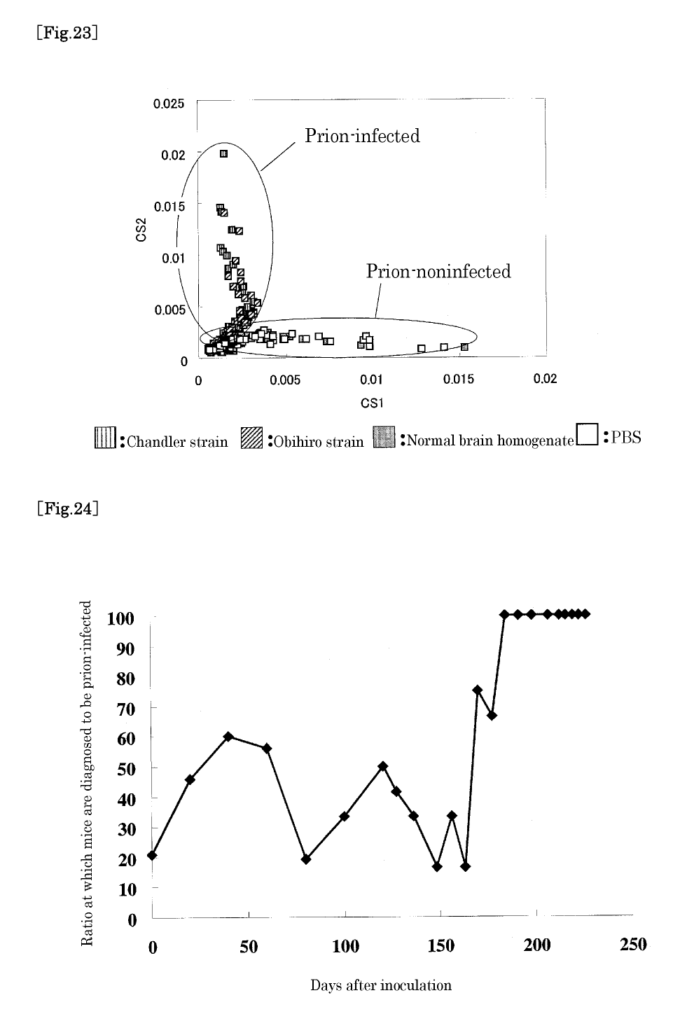

With respect to the spectral data obtained by the above-mentioned spectrometry, the SIMCA analysis was carried out with the same algorithm as in the first analysis method. Models for discriminating between prion infection and noninfection at least 170 days after inoculation were prepared, and a Coomans plot was examined. As shown in FIG. 23, models for discriminating between a group of Chandler-strain and Obihiro-strain prion-infected mice and a group of prion-noninfected mice inoculated with normal brain homogenate and PBS were able to be prepared.

Using the above-mentioned models, an examination was made about the ratio at which prion-infected mice subjected to spectrometry over time were diagnosed to be prion-infected with the models. As a result, as shown in FIG. 24, the ratio of the mice diagnosed to be prion-infected increased rapidly from about 160 days after inoculation, and eventually all the prion-infected mice were diagnosed to be prion-infected after about 180 days.

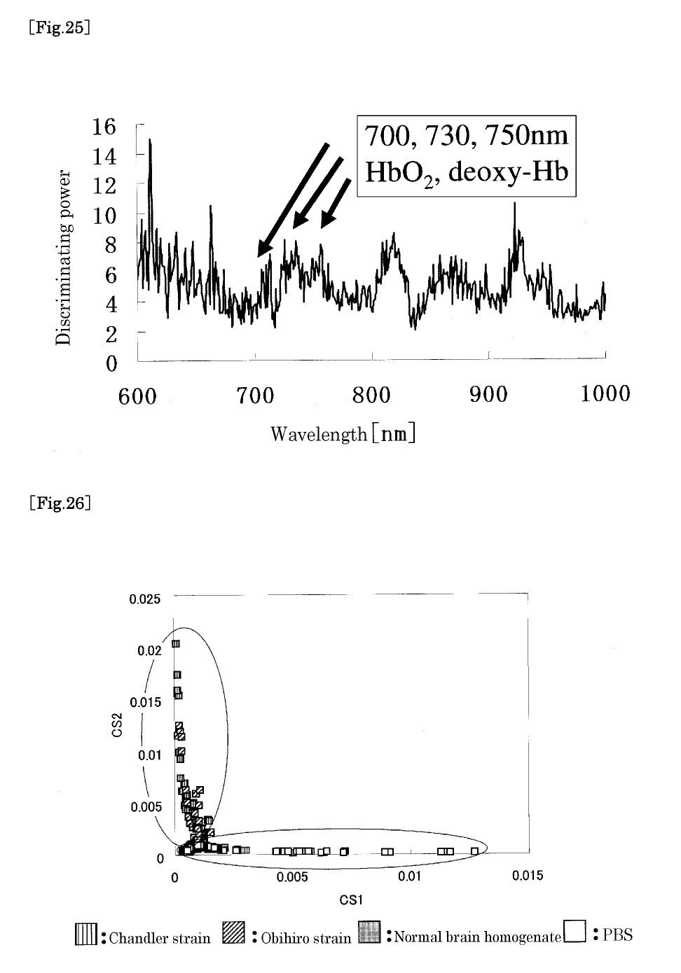

FIG. 25 shows the power of discriminating between prion infection and prion noninfection at each wavelength in the above-mentioned discrimination models obtained by the spectrometry carried out with the ears. It is of interest that peaks were found at wavelengths (700, 730, and 750 nm) associated with oxyhemoglobin (HbO₂) and deoxyhemoglobin (deoxy-Hb). Furthermore, as a result of the analysis, prion-inoculated mice had a lower concentration of oxyhemoglobin and a higher concentration of deoxyhemoglobin, as compared to those of the control mice. From these results, it can be judged that the above-mentioned discrimination model discriminates between prion infection and noninfection according to spectral data that reflect such a difference in biological bodies.

Next, the near-infrared spectrometry was carried out with respect to the abdomens in the same manner as in the above. In all the cases of intracerebral inoculation, intraperitoneal inoculation, and oral administration with respect to mice, once the amount of abnormal prion protein increases in a spleen, prion accumulates in the brain. Therefore reflected light was measured over time, with a fiber probe being placed against the abdomen of each of the aforementioned four types of mice. From the spectral data thus obtained, models for discriminating between prion infection and noninfection at least 170 days after inoculation were prepared, and a Coomans plot was examined. Accordingly, as shown in FIG. 26, models for discriminating between a group of Chandler-strain and Obihiro-strain prion-infected mice and a group of prion-noninfected mice inoculated with normal brain homogenate and PBS were able to be prepared.

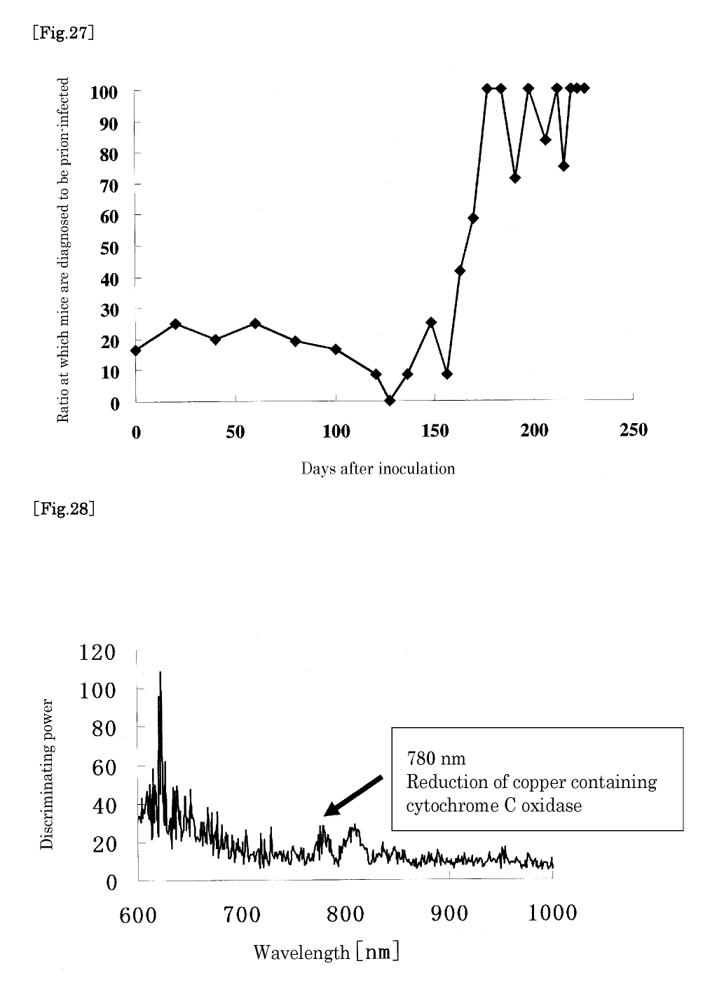

The above-mentioned models were used to examine the ratio at which prion-infected mice subjected to spectrometry over time were diagnosed to be prion-infected by using the models. As a result, as shown in FIG. 27, the ratio of the mice that were diagnosed to be prion-infected increased rapidly about 160 days after inoculation.

FIG. 28 shows the power of discriminating between prion infection and prion noninfection at each wavelength in the above-mentioned discrimination models obtained by spectrometry carried out with the abdomens. It is interesting that a peak was found at a wavelength (780 nm) associated with the reduction of copper containing cytochrome C oxidase. Furthermore, as a result of the analysis, it was proved that although the reduction of copper containing cytochrome C oxidase tended to decrease with the passage of days in the control mice, it increased on and after about 150 days in the prion-inoculated mice. From these results it can be judged that the above-mentioned discrimination model discriminates between prion infection and noninfection according to spectral data that reflect such a difference in biological bodies.

From the results described above, it was proved that an antemortem diagnosis of prion diseases by near-infrared spectroscopy could be made through measurements from the ears and abdomens of a biological body.

### Industrial Applicability

As described above, the present invention allows the presence of virus infection, such as HIV, and the presence of prion infection to be examined and judged simply and quickly with high accuracy. Thus it is widely applicable, for instance, to examination of virus infection and diagnosis of prion diseases.

1. A method of quantitatively or qualitatively examining and judging the presence of virus infection, such as HIV, or the presence of prion infection, the method comprising:
irradiating a sample derived from an examinee or other animal with light having a wavelength in a range of 400 nm to 2500 nm or a wavelength in part of the range,
detecting reflected light, transmitted light, or transmitted and reflected light to obtain an absorption spectral data, and
analyzing absorbance at all measurement wavelengths or at specific wavelengths in the absorption spectral data by using an analytical model prepared beforehand.

2. The examining and judging method according to claim 1, wherein a spectrum is measured, with a perturbation being provided for the sample by adding a predetermined condition, and an analysis that brings out an effect of the perturbation is performed.

3. The examining and judging method according to claim 2, wherein the predetermined condition is any one selected from the group consisting of change in concentration, repeated light irradiation, extension of irradiation time, addition of electromagnetic power, change in light path length, temperature, pH, pressure, mechanical vibration, and one that brings about physical or chemical change by changing the condition, or a combination thereof.

4. The examining and judging method according to claim 3, wherein light irradiation is repeated three times and thereby three absorption spectral data are obtained, and then a multivariate analysis is carried out by using at least two of the three absorption spectral data.

5. The examining and judging method according to claim 3, wherein with respect to one sample, those having a plurality of concentrations are prepared by dilution, they each are subjected to spectrum measurement, and a multivariate analysis is carried out by using the spectrum data thus obtained.

6. The examining and judging method according to claim 1, wherein the method is used for an HIV or prion test, and a target substance in a sample is quantified by using a quantitative model prepared by a regression analysis.

7. The examining and judging method according to claim 1, wherein the method is used for an HIV or prion test, and the presence of infection or a further infection period is estimated using a qualitative model prepared by a class discriminant analysis.

8. The examining and judging method according to claim 2, wherein the method is used for an HIV or prion test, and the presence of infection or a further infection period is estimated using a qualitative model prepared by (1) carrying out a regression analysis in which each value of perturbation is used as a dependent variable, and (2) carrying out a class discriminant analysis with respect to a regression vector obtained by the regression analysis.

9. The examining and judging method according to claim 1, wherein the wavelength region of the light with which the sample is irradiated is set in a range that is required for an analysis to be carried out using an analysis model.

10. The examining and judging method according to claim 9, wherein the wavelength region of the light with which the sample is irradiated is set in a range of 600 nm to 1000 nm.

11. The examining and judging method according to claim 1, wherein the sample is selected from the group consisting of blood, blood plasma, serum), urine, biological fluid, tissue, tissue extract, biological part, ear, abdomen, fingertip and toe tip.

12. A test and diagnostic device, comprising:
a floodlight means for irradiating a sample derived from an animal with light having a wavelength in a range of 400 nm to 2500 nm or a wavelength in part of the range,
a spectroscopic means for carrying out spectroscopy before or after irradiation and a detection means for detecting reflected light, transmitted light, or transmitted and reflected light with respect to the light with which the sample was irradiated, and
a data analysis means for analyzing qualitatively the presence of virus infection or the presence of prion infection by analyzing absorbance at all measurement wavelengths or at specific wavelengths in the absorption spectral data obtained by detecting, using an analytical model prepared beforehand.

13. The test and diagnostic device according to claim 12, wherein a spectrum is measured, with a perturbation being provided for the sample by adding a predetermined condition, and an analysis that brings out an effect of the perturbation is performed.

14. The test and diagnostic device according to claim 13, wherein the predetermined condition is any one selected from the group consisting of change in concentration, repeated light irradiation, extension of irradiation time, addition of electromagnetic power, change in light path length, temperature, pH, pressure, mechanical vibration, and one that brings about physical or chemical change by changing the condition, or a combination thereof.

15. The test and diagnostic device according to claim 14 wherein light irradiation is repeated three times and thereby three absorption spectral data are obtained, and a multivariate analysis is then carried out by using at least two of the three absorption spectral data.

16. The test and diagnostic device according to claim 12 wherein the test and diagnostic device is used for an HIV or prion test, and a target substance in a sample is quantified by using a quantitative model prepared by a regression analysis.

17. The test and diagnostic device according to claim 12, wherein the test and diagnostic device is used for an HIV or prion test, and the presence of infection or a further infection period is estimated using a qualitative model prepared by a class discriminant analysis.

18. The test and diagnostic device according to claim 13, wherein the test and diagnostic device is used for an HIV or prion test, and the presence of infection or a further infection period is estimated using a qualitative model prepared by (1) carrying out a regression analysis in which each value of perturbation is used as a dependent variable, and (2) carrying out a class discriminant analysis with respect to a regression vector obtained by the regression analysis.

19. The test and diagnostic device according to claim 27 wherein the wavelength region of the light with which the sample is irradiated is set in a range of 600 nm to 1000 nm.

20. The test and diagnostic device according to claim 12 wherein the sample is selected from the group consisting of blood, blood plasma, serum, urine, biological fluid, tissue, tissue extract, biological part, ear, abdomen, fingertip and toe tip.

21-25. (canceled)

26. The test and diagnostic device according to claim 12, further comprising a display means for displaying a result of an analysis.

27. The test and diagnostic device according to claim 12, wherein the wavelength region of the light with which the sample is irradiated is set in a range required for an analysis to be carried out using an analysis model.
